(19) United States
(12) Patent Application Publication
(10) Pub. No.: US 2026/0102616 A1
(43) Pub. Date: Apr. 16, 2026

(54) NEUROSTIMULATION SYSTEM AND METHODS

(71) Applicant: Encora, Inc., Boston, MA (US)

(72) Inventor: Daniel Carballo, Boston, MA (US)

(21) Appl. No.: 18/913,392

(22) Filed: Oct. 11, 2024

Related U.S. Application Data
(60) Provisional application No. 63/589,840, filed on Oct. 12, 2023.

(51) Int. Cl. A61N 1/36; A61H 23/02; A61N 1/372

(52) U.S. Cl. CPC A61N 1/36067; A61H 23/0254; A61N 1/36031; A61N 1/37223; A61H 2201/10; A61H 2201/105; A61H 2201/5012; A61H 2201/5097

(57) ABSTRACT

A neurostimulation system may include an implantable neurostimulator. An implantable neurostimulator may include a first sensor configured to sense internal data relating to a symptom of a neurological movement disorder of a patient. An implantable neurostimulator may include an internal stimulator configured to provide an internal stimulation output to an internal target area of a patient. An implantable neurostimulator may include a first wireless communication device configured to communicate internal data with a network. A neurostimulation system may include a wearable neurostimulator. A wearable neurostimulator may include a second sensor configured to sense external data of a patient. A wearable neurostimulator may include an external stimulator configured to provide an external stimulation output to an external target area of a patient. A wearable neurostimulator may include a second wireless communication device configured to communicate external data with a network and receive internal data from the network.

### CROSS REFERENCE TO RELATED APPLICATION

This application claims priority to U.S. Provisional Application No. 63/589,840, filed October 12, 2023, the entirety of both of which is incorporated herein by reference.

### TECHNICAL FIELD

The present disclosure relates to neurostimulation. In particular, the present disclosure relates to a neurostimulation system and methods.

### SUMMARY

In an aspect, a neurostimulation system is presented. A neurostimulation system may include an implantable neurostimulator. An implantable neurostimulator may include a first sensor configured to generate internal data relating to a symptom of a neurological movement disorder of a patient. An implantable neurostimulator may include an internal stimulator configured to provide an internal stimulation output to an internal target area of a patient. An implantable neurostimulator may include a first wireless communication device configured to communicate internal data with a network. An implantable neurostimulator may include a first processor in communication with a first sensor, internal stimulator, and first wireless communication device. A first processor may be configured to receive internal data from a first sensor and adjust an internal stimulation output based on the internal data. A neurostimulation system may include a wearable neurostimulator. A wearable neurostimulator may include a second sensor configured to generate external data of a patient. A wearable neurostimulator may include an external stimulator configured to provide an external stimulation output to an external target area of a patient. A wearable neurostimulator may include a second wireless communication device configured to communicate external data with a network and receive internal data from the network. A wearable neurostimulator may include a second processor in communication with a second sensor, external stimulator, and second wireless communication device. A second processor may be configured to revive external data from a second sensor and adjust external stimulation output based at least in part on internal data received from a network.

In another aspect, a method of neurostimulation is presented. A method may include sensing internal data of a patient by a sensor of an implantable neurostimulator. A method may include sensing external data of a patient by a sensor of a wearable neurostimulator. A method may include communicating internal data to a wearable neurostimulator and external data to an implantable neurostimulator via a network in communication with both the wearable neurostimulator and the implantable neurostimulator. A method may include generating one or more parameters of an internal stimulation output based on at least external data and one or more parameters of an external stimulation output based on at least internal data through a device in a network. A method may include stimulating either or both of an internal target area of a patient with internal stimulation output through an internal stimulator of an implantable neurostimulator and an external target area of the patient with an external stimulation output through an external stimulator of a wearable neurostimulator.

The above and other preferred features, including various novel details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein may be employed in various and numerous embodiments.

### BRIEF DESCRIPTION OF THE DRAWINGS

The disclosed embodiments have advantages and features which will be more readily apparent from the detailed description, the appended claims, and the accompanying figures(or drawings). A brief introduction of the figures is below.

FIG. 1 is an illustration of a system for neurostimulation according to embodiments described herein.

FIG. 2 is an illustration of a central architecture of the system shown in FIG. 1.

FIG. 3 illustrates an embodiment of a patient in combination with systems and methods described herein.

FIG. 4 illustrates a wearable device for neurostimulation according to embodiments described herein.

FIG. 5 illustrates an exploded view of the wearable device show in FIG. 4.

FIG. 6 is an illustration of an implantable neurostimulator, according to embodiments described herein.

FIG. 7 illustrates a flowchart of a method of neurostimulation according to embodiments described herein.

FIG. 8 is a block diagram of a machine learning module that may be implemented in systems and methods described herein.

FIG. 9 illustrates a computing device that may be implemented in systems and methods described herein.

### DETAILED DESCRIPTION

The Figures(Figs.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

Embodiments of the present disclosure may encompass both invasive and non-invasive neurostimulation for managing symptoms associated with a wide array of neurological disorders. These disorders include but are not limited to Parkinson's Disease, Dystonia, Essential Tremor, Epilepsy, Chronic Pain, Depression, and Obsessive-Compulsive Disorder (OCD). The embodiments described here allow for methodologies and systems for delivering precise electrical stimulation to specific neural targets associated with these disorders, utilizing either implanted devices or wearable neurostimulation devices. Through a meticulous orchestration of stimulation parameters, these embodiments may aim to alleviate symptoms and enhance the quality of life for individuals afflicted with these neurological disorders. Aspects of the present disclosure may be used to provide a combination of implantable and wearable neurostimulator stimulation. In some embodiments, aspects of the present disclosure may provide for various network architectures between implantable and wearable neurostimulators and/or one or more central and/or external computing devices.

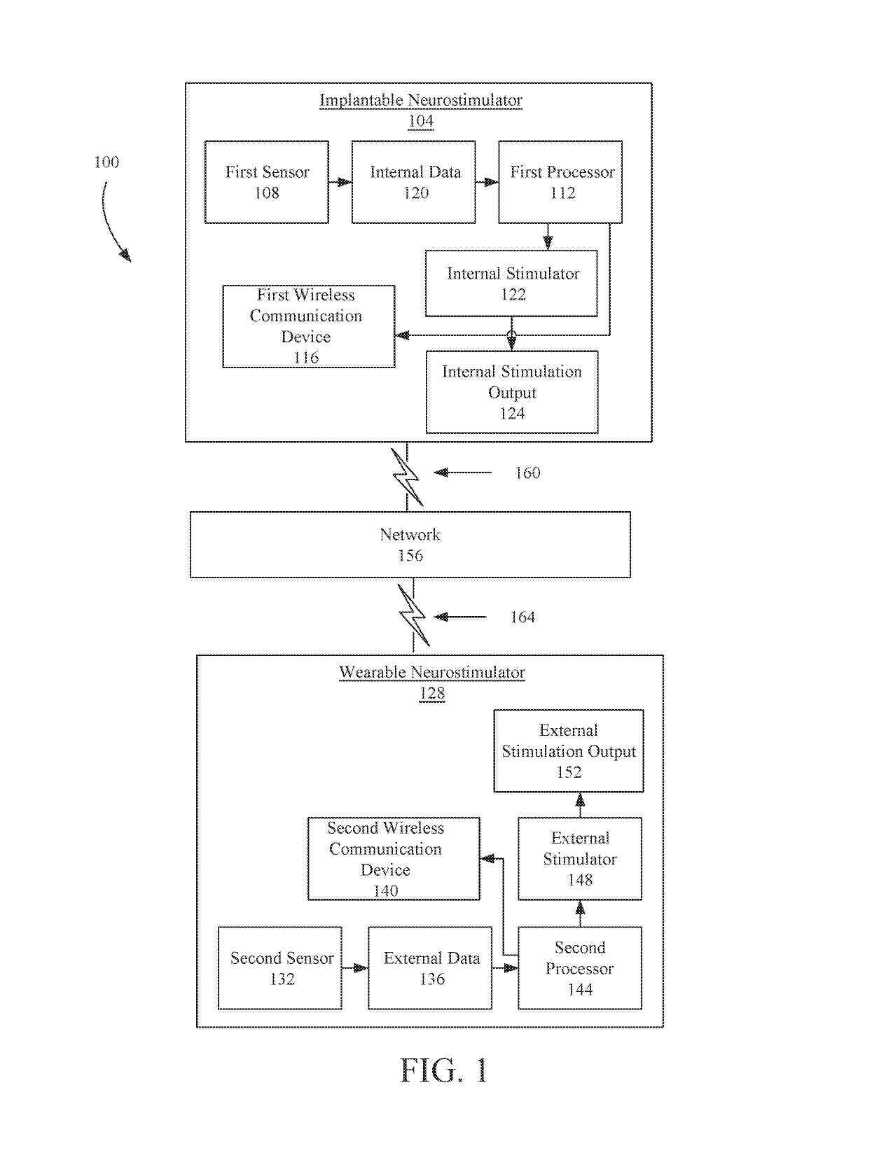

FIG. 1 illustrates a system 100 for neurostimulation in accordance with an embodiment of the present invention. System 100 may include implantable neurostimulator 104. An “implantable neurostimulator” as used in this disclosure refers to a device capable of applying stimulation to an internal target area from within a patient’s body. Implantable neurostimulator 104 may include, but is not limited to, a deep brain stimulator (DBS), spinal cord stimulator, vagus nerve stimulator, and/or other types of implantable neurostimulators. Implantable neurostimulator 104 may be implantable into a skull of a patient, a chest of a patient, a back of a patient, a stomach of a patient, and/or other areas of a patient’s body. In some embodiments, implantable neurostimulator 104 may include first sensor 108. A “sensor” as used in this disclosure refers to a device capable of detecting changes in physical properties. Physical properties may include, but are not limited to, electromagnetic fields, chemical compositions, currents, voltages, physical forces, and/or other physical properties. In some embodiments, first sensor 108 may be configured to generate internal data 120 of an internal target area of a patient. An “internal target area” as used in this disclosure refers to a portion of an inside of a patient’s body. Internal target areas may include, but are not limited to, tissues, neural structures, nerves, and/or other parts of a patient’s body. For instance and without limitation, implantable neurostimulator 104 may be surgically embedded into a patient’s chest cavity and connected via insulated leads to electrodes implanted in deep brain regions associated with motor control such as the Subthalamnic Nucleus (STN) or Globus Pallidus Interna (GPi).

First sensor 108 may be an electroencephalography (EEG) sensor, electromyography (EMG) sensor, local field potential sensor, and/or other type of sensor. First sensor 108 may be configured to sense data and/or generate internal data 120. Internal data 120 may relate to a symptom of a neurological movement disorder of a patient such as, but not limited to, Parkinson’s, Essential Tremor, or other neurological movement disorders. First sensor 108 may generate internal data 120 based on one or more detected properties of one or more parts of an inside of a patient’s body. “Internal data” as used in this disclosure refers to physical properties measured from inside a patient’s body. Internal data 120 may include, but is not limited to, current values, voltage values, electrical waveforms such as alternating current (AC) values, direct current (DC) values, and/or other values. Internal data 120 may include brain waveforms, activation of neurons in specific regions of a brain, and/or other data. In some embodiments, implantable neurostimulator 104 may have first processor 112. First processor 112 may include a system on chip (SoC), microcontroller, microprocessor, or other computing device. First processor 112 may be in electrical communication with first sensor 108. For instance, first sensor 108 may be electrically wired to first processor 112. First processor 112 may be configured to receive sensor data generated from first sensor 108, such as internal data 120, and generate internal stimulation output 124. “Internal stimulation output” as used in this disclosure refers to stimulation provided to an internal target of a patient’s body. Internal stimulation output 124 may have one or more parameters and/or values. For instance and without limitation, internal stimulation output 124 may have one or more currents, voltages, frequencies, and/or other values. Internal stimulation output 124 may be provided by internal stimulator 120. An “internal stimulator” as used in this disclosure refers to a stimulation device that is capable of outputting stimulation to an internal target of a patient’s body. Internal stimulator 120 may include, but is not limited to, electrodes, transformers, and/or other devices. First processor 112 may be in electrical communication with internal stimulator 120 and may be configured to active internal stimulator 120 to produce internal stimulation output 124 to an internal target area of a patient’s body. In some embodiments, implantable neurostimulator 104 may include a digital to analog converter that may be configured to convert parameters of internal stimulation output 124 generated by first processor 112, such as digital stimulation settings of internal stimulation output 124, into signals usable by internal stimulator 120 to produce internal stimulation output 124, such as analog voltage values. In some embodiments, implantable neurostimulator 104 may include various circuitry and/or electrical components such as, but not limited to, voltage multipliers, current regulators, and/or other electrical components that may assist in providing internal stimulation output 124. First processor 112 may be configured to run a real-time operating system, such as, but not limited to, FreeRTOS. First processor 112 may utilize one or more optimization algorithms for efficient coding. For instance, the Intel Math Kernel Library (MKL) may be used. First processor 112 may utilize AVX2 vector extensions on x86 architecture. In some embodiments, the GNU Scientific Library may be used, which may supply optimized routines for statistical computations including random number generation, interpolation, integration, and/or differentiation, which may reduce a number of CPU cycles needed for calculations. First processor 112 may be configured to perform parallel processing, multithread processing, and/o rother processing techniques.

Referring still to FIG. 1, first processor 112 may be configured to calculate internal stimulation output 124 based on internal data 120. For instance and without limitation, internal stimulation output 124 may include an electrical waveform, electromagnetic waveform, laser waveform, or other waveform. First processor 112 may be configured to adjust parameters such as, but not limited to, amplitude, frequency, pulse width, voltages, currents, and/or other parameters of internal stimulation output 124. First processor 112 may be operable to run firmware and/or software. Firmware and/or software may be loaded onto a memory unit of implantable neurostimulator 104. Memory units may include, but are not limited to, dynamic random-access memory (DRAM), random-access memory (RAM), and/or solid state drives (SSD), and/or any other type of memory storage device. First processor 112 may be operable to execute firmware and/or software instructions stored in one or more memory units. In some embodiments, memory units may be pre-loaded with parameters and/or other settings of internal stimulation output 124 based on clinical data of an application of implantable neurostimulator 104. For instance, parameters and/or other settings of internal stimulation output 124 may be applicable to reducing movement disorder symptoms of a patient. Clinically-derived parameters of internal stimulation output 124 may be loaded onto one or more memory units of implantable neurostimulator 104 and may be used by first processor 112 to generate internal stimulation output 124.

In some embodiments, implantable neurostimulator 104 includes first wireless communication device. A “wireless communication device” as used herein refers to a device capable of sending and receiving electromagnetic signals. First wireless communication device 116 may include, but is not limited to, Bluetooth modules, Wi-Fi modules, cellular modules, Zigbee modules, and/or other types of wireless communication devices. In some embodiments, first wireless communication device 116 may include a Bluetooth Low Energy module. First wireless communication device 116 may be configured to communicate with network 156 via first link 160. First link 160 may be a Wi-Fi, Bluetooth, or other connection. A “network” as used in this disclosure is a communication link between two or more computing devices. In some embodiments, first wireless communication device 116 may be configured to communicate internal data 120, operational data of internal stimulator 120, and/or parameters of internal stimulation output 124 to network 156. In some embodiments, network 156 may communicate sensor data and/or parameters of internal stimulation output 124 to first wireless communication device 116. First processor 112 may be configured to adjust internal stimulation output 124 based on data received by network 156. First link 160 may operate within 2.4GHz, 5GHz, or other frequencies. First link 160 may operate at a transmission power of about -20dBm to about 10dBm. In some embodiments, first link 160 may have a transmission power of lower than about -20dBm or greater than about 10dBm. First link 160 may operate in intervals of between about 7.5ms to about 4s. In some embodiments, first link 160 may operate in intervals of less than about 7.5ms or greater than about 4s.

Implantable neurostimulator 104 may include a power source, which may include a lithium-ion or other battery, without limitation. In some embodiments, a power source of implantable neurostimulator 104 may be rechargeable via a USB, micro-USB, USB-type C, and/or inductive charging. A power source may have up to or greater than a 10 hour battery life, in some embodiments. Implantable neurostimulator 104 may include charging circuitry that may manage safe recharging of a power source via wireless induction. In some embodiments, while a power source is being wireless recharging, communication with external devices may be facilitated by wireless communication protocols such as, but not limited to, near-field communication (NFC) or Medical Implant Communication System (MICS). Communication with external devices while a power source of implantable neurostimulator 104 is recharging may enable a monitoring of charging status and/or an adjustment of one or more stimulation parameters.

In some embodiments, implantable neurostimulator 104 may include a single-channel implantable pulse generator (IPG). In embodiments where implantable neurostimulator 104 may be an IPG, implantable neurostimulator 104 may include a housing encapsulated within a biocompatible casing such as, but not limited to, titanium. A housing may store first processor 112, first sensor 108, internal stimulator 120, first wireless communication device 116, one or more memory units, and/or a power source. In embodiments where implantable neurostimulator 104 includes a single-channel IPG, internal stimulator 120 may include one or more electrodes. In some embodiments, implantable neurostimulator 104 may include a multi-channel IPG. A multi-channel IPG may include a plurality of electrode channels, each with their own stimulation parameters which may be independently adjustable. In embodiments where neurostimulator 104 includes a multi-channel IPG, internal stimulator 120 may include a plurality of electrodes. In some embodiments, implantable neurostimulator 104 may be miniaturized. For instance, first processor 112 may include one or more application-specific integrated circuits (ASICs) which may help minimize a size and weight of implantable neurostimulator 104 as well as power consumption of implantable neurostimulator 104.

With continued reference to FIG. 1, in some embodiments, implantable neurostimulator 104 may include a drug delivery system. For instance, implantable neurostimulator 104 may include one or more micro-pumping mechanism which may allow for the delivery of precise drug dosages. Drug delivery may occur additionally or alternatively to internal stimulation output 124. Implantable neurostimulator 104 may include a reservoir that may be refillable. A reservoir of implantable neurostimulator 104 may include medicaments such as, but not limited to, carbidopa, levodopa, propranolol, primidone, gabapentin, topiramate, or other medicaments. A reservoir of implantable neurostimulator 104 may be in fluidic communication with one or more fluid conduits, such as, but not limited to, tubes, needles, cannulas, or other fluid delivery devices. First processor 112 may be in electric communication with a micro-pump or other drug delivery mechanism and may be configured to control doses, dose timing, and/or other delivery of a medicament in a reservoir of implantable neurostimulator 104. In some embodiments, first processor 112 may be configured to activate one or more micro-pumping mechanisms based on internal data 120 and/or external data 136. For instance and without limitation, in response to internal data 120 and/or external data 136, first processor 112 may be instruct or otherwise command a micro-pump of implantable neurostimulator 104 to deliver one or more medicaments of a reservoir of implantable neurostimulator 104 to one or more internal target areas of a patient’s body, such as, but not limited to, regions of the patient’s brain, directly into a central nervous system (CNS) of the patient, into a spinal cord of a patient, into cerebrospinal fluid of a patient, or other areas. A fluid delivery device, such as a needle and/or cannula, may be placed at internal target areas of a patient’s body. In some embodiments, implantable neurostimulator 104 may include a plurality of fluid delivery devices that may correspond to a particular medicament and/or internal target area. For instance and without limitation, a first fluid delivery device may be placed at a particular region of a patient’s brain, while a second fluid delivery device may be placed at a position in the patient’s spinal column. Based on internal data 120 and/or external data 136, first processor 112 may activate one or more micro-pumps to deliver a first medicament to a first internal target area of a patient’s body and a second medicament to a second internal target area of the patients body.

In some embodiments, implantable neurostimulator 104 may be modular. Implantable neurostimulator 104 may include a base module, which may house first processor 112, power management circuitry, and/or wireless communication circuitry. One or more additional modules may be connectable or otherwise attachable to a base module of implantable neurostimulator 104. For instance one or more parts of implantable neurostimulator 104 may be configured to be removed and/or swapped with one or more other parts of implantable neurostimulator 104. Additional first sensors 108 may be added, different types of first sensors 108 may be added, and/or one or more first sensors 108 may be removed. Different types of first processors 112 may be added, removed, or swapped. Likewise, different types of internal stimulators 120 and/or first wireless communication devices 116 may be added, removed, or swapped. Firmware of implantable neurostimulator 104 may be configured to allow for a changing of one or more parts and/or modules of implantable neurostimulator 104 while ensuring proper functionality of implantable neurostimulator 104. Various arrangements of modular parts of implantable neurostimulator 104 may be assembled that may be specific to a patient and/or disease.

Referring still to FIG. 1, in some embodiments, system 100 includes wearable neurostimulator 128. A “wearable neurostimulator” as used in this disclosure refers to a stimulation device capable of securing itself to an external part of a patient’s body. Wearable neurostimulator 128 may include, but is not limited to, wrist-worn devices, headbands and/or helmet devices, vests and/or shirt devices, patch devices, ankle or foot worn devices, belt devices, and/or glove devices.

Wearable neurostimulator 128 may include second sensor 132. Second sensor 132 may include, but is not limited to, electrical sensors such as current and/or voltage sensors, temperature sensors, skin conductivity sensors, accelerometers, gyroscopes, MEMS-based accelerometers, magnetometers, inertial measurement units, and/or other sensors. For instance and without limitation, second sensor 132 may include a 6-axis IMU chip, which may integrate a 3-axis accelerometer and 3-axis gyroscope. Second sensor 132 may be configured to sense and/or generate external data 136. “External data” as used in this disclosure refers to a physical property measured from outside of a patient’s body. External data 136 may relate to one or more symptoms of a neurological movement disorder of a patient, such as, but not limited to, Parkinson’s, Essential Tremor, or other neurological movement disorders. For instance and without limitation, external data 136 may include, but is not limited to, accelerometer values, gyroscope values, skin conductivity values, voltages, currents, and/or other values. Wearable neurostimulator 128 may include second processor 144. Second processor 144 may be in electrical communication with second sensor 132 and may be configured to receive external data 136 from second sensor 132. Second processor 144 may be a SoC, microcontroller, microprocessor, and/or other computing device. In some embodiments, second processor 144 may be a same type of processor as first processor 112. In other embodiments, second processor 144 may be a different type of processor than first processor 112. Second processor 144 may be configured to run a real-time operating system, such as, but not limited to, FreeRTOS. Second processor 144 may utilize one or more optimization algorithms for efficient coding. For instance, the Intel Math Kernel Library (MKL) may be used. Second processor 144 may utilize AVX2 vector extensions on x86 architecture. In some embodiments, the GNU Scientific Library may be used, which may supply optimized routines for statistical computations including random number generation, interpolation, integration, and/or differentiation, which may reduce a number of CPU cycles needed for calculations. Second processor 144 may be configured to perform parallel processing, multithread processing, and/o rother processing techniques.

Second processor 144 may be configured to receive external data 136 and generate external stimulation output 152. “External stimulation output” as used herein refers to stimulation provided to an external target area of a patient’s body from outside the patient’s body. An “external target area” as used in this disclosure refers to an external portion of a patient’s body selected for stimulation. External portions of a patient’s body may include, but are not limited to, nerves and/or tissues of wrists, ankles, chest, necks, hands, and/or other external parts of a patient’s body. External stimulation output 152 may be provided by external stimulator 148. An “external stimulator” as used in this disclosure refers to a device capable of providing stimulation output from an outside of a patient’s body. External stimulator 148 may include, but is not limited to, electrodes, vibratory motors, heating elements, ultrasonic emitters, and/or other types of stimulators. Second processor 144 may be in communication with external stimulator 148 and may be configured to active or otherwise command external stimulator 148 to provide external stimulation output 152. In some embodiments, external stimulation output 512 may be a stimulation waveform. A stimulation waveform may include one or more parameter such as, but not limited to, frequencies, pulse widths, amplitudes, and/or other values. Second processor 144 may be configured to adjust and/or calculate any stimulation parameters of external stimulation output 152. Second processor 144 may calculate and/or generate stimulation parameters of external stimulation output 152 based on external data 136. Second processor 144 may be configured to run a real-time operating system, such as, but not limited to, FreeRTOS.

Second processor 144 may be configured to adjust parameters such as, but not limited to, amplitude, frequency, pulse width, voltages, currents, and/or other parameters of external stimulation output 152. Second processor 144 may be operable to run firmware and/or software. Firmware and/or software may be loaded onto a memory unit of wearable neurostimulator 128. Memory units may include, but are not limited to, dynamic random-access memory (DRAM), random-access memory (RAM), and/or solid state drives (SSD), and/or any other type of memory storage device. Second processor 144 may be operable to execute firmware and/or software instructions stored in one or more memory units. In some embodiments, memory units may be pre-loaded with parameters and/or other settings of external stimulation output 152 based on clinical data of an application of implantable neurostimulator 104. For instance, parameters and/or other settings of external stimulation output 128 may be applicable to reducing movement disorder symptoms of a patient. Clinically-derived parameters of external stimulation output 152 may be loaded onto one or more memory units of wearable neurostimulator 128 and may be used by second processor 144 to generate external stimulation output 152. In some embodiments, wearable neurostimulator 128 may include an attachment mechanism. An attachment mechanism may include, but is not limited to, straps, hoops, locks, Velcro, shirts, belts, headbands, socks, and/or other attachment mechanisms.

In embodiments where wearable neurostimulator 128 may be a wrist-worn device, wearable neurostimulator 128 may include a wrist-band or other device. External stimulator 148 may integrate an array of transcutaneous electrical nerve stimulations (TENS) electrodes embedded into an underside of a watch band of wearable neurostimulator 128. Electrodes may be fabricated from biocompatible materials such as, but not limited to, stainless steel or conductive hydrogel. Wearable neurostimulator 128 may provide external stimulation output 152 in the form of TENS stimulation with parameters such as, but not limited to, pulse width, amplitude, and/or frequency. Pulse widths of TENS stimulation may be between about, but not limited to, 50-200 μs, Amplitudes of TENS stimulation may be up to or greater than 80V in some embodiments. Frequencies of TENS stimulation may range from between 10Hz to 150Hz. Second processor 144 may be configured to adjust any parameter of TENS stimulation, without limitation. In some embodiments, external stimulator 148 may include one or more vibratory motors. External stimulator 148 may include a combination of TENS electrodes and one or more vibratory motors, in some embodiments. Vibratory motors may include eccentric mass vibration motors. Vibratory motors of external stimulator 148 may be configured to produce vibratory haptic cues at frequencies from about 50Hz to about 280 Hz, without limitation.

In embodiments where wearable neurostimulator 128 may be a headband or helmet device, wearable neurostimulator 128 may include a lightweight and/or adjustable polymer housing of a head strap, headband, or other device. Wearable neurostimulator 128 may include a power source, such as a lithium-ion or other type of battery. A power source may be rechargeable via a USB, USB-type C, mircoUSB, and/or inductive charging. Wearable neurostimulator 128 may be made of a lightweight and/or adjustable polymer. External stimulator 148 may include an array of electrodes that may be integrated into a headband or liner of a helmet to make scalp contact, which may provide for external stimulation output 152 of transcranial direct current stimulation (tDCS) and/or transcranial alternating current stimulation (tACS). tDCS and/or tACS provided by external stimulator 148 may target internal target areas of a patient’s body, such as brain regions like the motor cortex, without limitation. Electrodes of external stimulator 148 may be made form a conductive rubber or hydrogel-based material. Stimulation waveform parameters in embodiments where wearable neurostimulator 128 may be a headband or helmet device may include, but are not limited to, a current amplitude of about 1 mA to about 2mA, a frequency of about 0.5Hz to about 1KHz, and/or phase and polarity values. External stimulator 148 may alternatively or additionally include one or more vibratory motors, such as eccentric rotating mass vibration motors which may be placed along a forehead region. Additionally or alternatively, external stimulator 148 may include sound haptic actuators, which may produce a sonic output between about 180Hz to about 320Hz. In embodiments where wearable neurostimulator 128 may be a headband or helmet device, second sensor 132 may include one or more EEG sensor spanning frontal, motor, parietal, and/or occipital areas. One or more EEG sensors may be configured to monitor brain dynamics in real-time. One or more EEG sensors may include gel-based electrodes. Data generated by EEG sensors in embodiments where wearable stimulator 128 may be a helmet or headband may be utilized by second processor 144 to device brain states, detect signatures of worsening symptoms, and/or provide neurofeedback. For instance in some embodiments, second processor 144 may be configured to run an on-board machine learning algorithm. A machine learning algorithm may be trained with training data correlating EEG sensor data to brain states, predicted worsening symptoms, and/or other parameters. Training data may be received from user input, external computing devices, and/or previous iterations of processing.

In embodiments where wearable neurostimulator 128 may be a helmet or headband device, second sensor 132 may include one or more motion sensors such as gyroscopes and/or magnetometers. Motion sensors may enable second processor 144 to track head movements, which may allow for analyses of conditions such as, but not limited to, cervical dystonia. Second processor 144 may be configured to adaptively adjust stimulation parameters of external stimulation output 152 based on head movements of a patient.

In embodiments where wearable neurostimulator 128 may be a vest or shirt device, external stimulator 148 may include one or more electrodes. Electrodes and/or muscle activity sensors may be embedded into a fabric of a vest or shirt device. In some embodiments, electrodes of a vest or shirt device may be arranged to target torso, chest, abdomen, and/or back muscles and/or tissues of a patient. External stimulator 148 may include an array of silicone-encapsulated stainless steel pad electrodes, which may be integrated into a fabric to contact with a skin surface of a patient. External stimulator 148 may be configured to provide external stimulation output 152 in a form of TENS or interferential stimulation. One or more parameters of external stimulation output 152 may be configured to module pain signals and/or improve mobility in a patient. Parameters may include, but are not limited to, amplitude, burst frequency, pulse width, and/or other parameters. Amplitudes may be between about 20mA to about 80mA, pulses may include biphasic pulses with a pulse width of about 200 μs, and frequencies may range from about 2Hz to about 120Hz. Second processor 144 may be configured to adjust any parameter of external stimulation output 152. Additionally or alternatively, external stimulator 148 may include one or more vibratory motors, such as, but not limited to, eccentric rotating mass vibration motors. Vibratory motos may be stitched into a garment lining at locations corresponding to major muscle groups. Vibratory motors may provide an external stimulation output 152 of a vibration intensity between about 60Hz to about 240Hz.

In embodiments where wearable neurostimulator 128 may be a vest or shirt device, second sensor 132 may include one or more inertial sensors and/or EMG sensors. Second processor 144 may be configured to receive EMG data and determine predicted worsening symptoms, compensation behaviors, or other data, and may adjust external stimulation output 152 to accommodate for the determinations made. For instance and without limitation, an increase in an EMG spike in back muscle may trigger escalating electrical stimulation to counteract building spasticity before pain or injury results. Second sensor 132 may include inertial sensors such as accelerometers and/or gyroscopes that may be distributed through a garment and may quantitatively track body movements of a patient. Second processor 144 may be configured to run an on-board machine learning model that may input EMG and/or inertial sensor data and may output one or more stimulation parameters of external stimulation output 152. A machine learning model may be trained with training data correlating EMG and/or inertial data to one or more parameters of external stimulation output 152. Training data may be received via user input, external computing devices, and/or previous iterations of processing. Electrodes and/or sensors of a vest or shirt device may be embedded and/or integrated into a fabric of the vest or shirt device, which may allow the vest or shirt device to be washable. For instance, electrodes and/or sensors may be partially or fully encapsulated by an insulating coating, such as a rubber.

In embodiments where wearable neurostimulator 128 may be a patch device, wearable neurostimulator 128 may include an adhesive patch form. External stimulator 148 may include one or more embedded electrodes, vibratory motors, and/or ultrasonic emitters in an adhesive patch. Electrodes may be hydrogel electrodes with conductive carbon or silver chloride coatings and may be configured to provide TENS. Parameters of TENS in a patch form may include, but is not limited to, a pulse width of about 30 to about 240 μs, an amplitude of about 10mA to about 100mA, and/or a frequency of about 2Hz to about 150Hz. Second processor 144 may be configured to adjust any parameter of TENS. In some embodiments, external stimulation output 152 may include vibratory output. Vibratory motors may provide one or more mechanical vibrations with a frequency of about 100Hz to about 280Hz. In some embodiments, external stimulation output 152 may include ultrasonic output. External stimulator 148 may include a piezoelectric MEMS transducer with a resonant frequency of about 1MHz to about 5MHz. Amplitude, duty cycle, and/or frequency of an ultrasonic output may be adjusted by second processor 144.

In embodiments where wearable neurostimulator 128 may be an ankle or foot-word device, wearable neurostimulator 128 may be integrated into shoes and/or socks. For instance, external stimulator 148 may include an array of electrodes lining a footbed or sock that may be configured to provide TENS to different external target areas of a patients body involved in proprioception and/or motor control. Additionally or alternatively, external stimulator 148 may include one or more vibratory motors, such as vibration haptic actuators like voice coils, which may be embedded along a sock lining or shoe insole. In some embodiments, external stimulator 148 may include one or more ultrasonic transducers arranged along an ankle band which may provide focused ultrasound stimulation of lower leg nerves and/or muscles of a patient. Second processor 144 may be configured to adjust TENS, vibratory stimulation, and/or ultrasonic stimulation based on real-time gait dynamics of a patient measured by second sensor 132. For instance, second sensor 132 may include one or more inertial measurement devices such as gyroscopes and/or accelerometers. Second processor 144 may be configured to run an on-board machine learning model which may sensor data and may output one or more parameters of external stimulation output 152. A machine learning model may be trained to detect gait anomalies from data generated by second sensor 132 and trigger preemptive stimulation interventions to counteract building imbalance through phase-specific sensory augmentation via external stimulation output 152.

In embodiments where wearable neurostimulator 128 may be a belt device, wearable neurostimulator 128 may be integrated into a belt material. For instance, external stimulator 148 may include a ring of silicone-encapsulated electrodes embedded in a belt lining and configured to provide TENS to various external target areas, such as, but not limited to, lower back nerves. Second processor 148 may be configured to adjust an intensity of external stimulation output 152 in real time based on a patient’s posture and/or movements detected by second sensor 132, which may include one or more integrated movement sensors. TENS parameters provided in a belt form may include, but are not limited to, about 100Hz frequency, about 200 μs pulse width, and amplitudes up to 100 mA, all of which may be controlled in real time by second processor 148. Additionally or alternatively, external stimulator 148 may include one or more vibratory motors, such as, but not limited to, eccentric rotating mass vibration motors. Vibratory motors may be stitched into a belt lining and arranged to provide mechanical vibratory stimulation to an external target area of a lumbar region of a patient. Mechanical vibratory stimulation may include an intensity of about 60 Hz to about 240Hz. External stimulation output 152 may include vibratory output, which may enhance muscle activation in a patient with stimulation delivery timed based on EMG patterns detected by second sensor 132.

Additionally or alternatively, external stimulator 148 may include one or more ultrasonic transducers. Ultrasonic transducers may be configured to output a frequency of up to or greater than about 5MHz. Second sensor 132 may include one or more EMG sensors, accelerometers and/or gyroscopes distributed and/or embedded along a belt, which may be configured to quantify back movements of a patient. In some embodiments, one or more sensor fusion algorithms may be employed. Sensor fusion algorithms may analyze spine curvature, gait phase transitions, posture sway, and/or ergonomics which may be provided to second processor 144. Sensor fusion and/or signal processing algorithms may be used to analyze EMG data to decode muscle fatigue, coordination impairments, and/or co-contraction of one or more muscles of a patient. Second processor 144 may be configured to adjust external stimulation output 152 based on data generated by one or more sensor fusion algorithms. For instance, external stimulation output 152 may be adjusted to active one or more muscles of a patient reflexively to restore neutral posture and offload one or more discs of the patient. Second processor 144 may be configured to run an on-board machine learning model. A machine learning model may be trained to input sensor data and output one or more parameters of external stimulation output 152. Training data may be received via user input, external computing devices, and/or previous iterations of processing. A machine learning model may be configured to input sensor data and detect early signs of fatigue or pain based on movement pattern changes determined through data generated by second sensor 132.

In embodiments where wearable neurostimulator 128 may be a glove device, wearable neurostimulator 128 may be formed as a glove. For instance, one or more sensors and/or electrodes may be mounted along glove fingertips and/or glove lining. External stimulator 148 may include an array of electrodes mounted on one or more glove fingertips and/or glove lining and configured to provide TENS to one or more nerves of hands and/or wrists of a patient. Electrode material may include a biocompatible conductive polymer, hydrogel, and/or other material. Additionally or alternatively, external stimulator 148 may include one or more vibratory motors, such as, but not limited to, eccentric rotating mass vibration motors. One or more vibratory motors may be embedded along one or more fingertips of a glove. One or more vibratory motors may be configure to provide supplementary vibratory sensor stimulation coordinate with TENS patterns. Vibratory motors may produce vibratory stimulation having a frequency of about 100Hz to about 280Hz. Second sensor 132 may include one or more inertial sensors such as, but not limited to, accelerometers and/or gyroscopes which may be distributed across one or more glove joints. One or more sensor fusion algorithms may be employed to analyze motion data in real-time which may allow second processor 144 to determine metrics such as, but not limited to, joint coordination, grip strength, range of motion, and/or other metrics. Second processor 144 may be configured to run an on-board machine learning model that may be trained to input sensor data and output one or more parameters of external stimulation output 152. Training data may be received via user input, external computing devices, and/or previous iterations of processing. Second sensor 132 may include one or more EMG sensors fabricated from conductive threads and integrated along a glove, which may allow for monitoring of muscle activity in real-time. One or more signal processing algorithms may be employed, which may allow second processor 144 to decode gestures and/or dexterity. In some embodiments, a glove device may include one or more user input buttons and/or touch interfaces which may allow a patient to adjust power, sensor, and/or stimulation settings.

Still referring to FIG. 1, wearable neurostimulator 128 may include second wireless communication device 140. Second wireless communication device 140 may include, but is not limited to, a Bluetooth module, Wi-Fi module, cellular module, and/or other type of wireless communication device. In some embodiments, second wireless communication device 140 may be a same type of wireless communication device as first wireless communication device 116. In other embodiments, second wireless communication device 140 may be a different type of wireless communication device than that of first wireless communication device 116. Second wireless communication device 140 may be configured to communicate with network 156. In some embodiments, second wireless communication device 140 may be configured to communicate external data 136, operational data of external stimulator 148, and/or parameters of external stimulation output 152 with network 156 via second link 164.

In some embodiments, network 156 may be between implantable neurostimulator 104 and wearable neurostimulator 128. For instance, wearable neurostimulator 128 may be configured to directly communicate with implantable neurostimulator 104. A direct communication between implantable neurostimulator 104 and wearable neurostimulator 128 may be referred to herein as a “Direct Communication Architecture.” A direct communication between implantable neurostimulator 104 and wearable neurostimulator 128 may occur over network 156, which take form of Bluetooth at 2.5GHZ and/or 5GHz, Bluetooth low energy, Thread, Zibgee, and/or other protocols. In some embodiments, a radio frequency (RF) protocol may be implemented on each of first wireless communication device 116 and second wireless communication device 140. A direct communication between implantable neurostimulator 104 and wearable neurostimulator 128. Network 156 between implantable neurostimulator 104 and wearable neurostimulator 128 may be encrypted. Encryption may include, but is not limited to, 128-bit AES, 256-bit AES, or other encryption methods. Each of implantable neurostimulator 104 and wearable neurostimulator 128 may have one or more hardware acceleration modules that may be used to secure a direct link between both devices. Authentication mechanisms, such as device-specific key exchange may be implemented between implantable neurostimulator 104 and wearable neurostimulator 128. Specific key exchange mechanisms may use Elliptic Curve Diffie-Hellman encryption.

In some embodiments, first link 160 and/or second link 164 may be operated under an error detection and/or correction mechanism to ensure high reliability and integrity of communication between implantable neurostimulator 104, wearable neurostimulator 128, and network 156. Techniques such as Cyclic Redundancy Check (CRC) may be used. In CRC, a CRC code may be generated based on a data payload and may be appended to aa packet before transmission. A receiver may recalculate a CRC code and compare it to a received CRC code to detect any errors. A CRC generator may be used, in which the CRC generator creates a CRC code having a polynomial and code length, which may be optimized to maximize error detection capability for packet sizes and data throughput. For instance and without limitation, a 16-bit CRC code may be implemented using a CRC-16-CCITT polynomial.

Techniques such as Forward Error Correction (FEC) may be employed. FEC may use redundant parity data that may be encoded into a packet to allow recover of errors at a receiver without retransmission. FEC schemes such as Reed-Solomon and/or convolutional codes may be implemented. Code rates may be adapted base don channel noise characteristics. For instance and without limitation, a Reed-Solomon (255223) code may provide up to 16 byte error correction capability per block. Packet combining methods like incremental redundancy or Chase combing may be employed. In some embodiments, implantable neurostimulator 104 may include a dedicated FEC encoder/decoder hardware accelerator which may allow for low-latency error correction. An FEC scheme may additionally or alternatively be implemented in software libraries such as KISS or channel codes on one or more microcontrollers of implantable neurostimulator 104, such as first processor 112. Channel noise characteristics may be analyzed through simulations and/or channel sounding measurements. Tolerance to residual errors after decoding may be evaluated for different traffic types, such as telemetry data versus closed loop control data. Network layer retransmission protocols like ARQ may be additionally incorporated to ensure zero error tolerance for critical data.

In some embodiments, pairing between implantable neurostimulator 104 and wearable neurostimulator 128 may occur. For instance, first processor 112 and/or second processor 144 may perform a pairing and/or authentication process through data communicated via network 156 between implantable neurostimulator 104 and wearable neurostimulator 128. A pairing request may be sent from wearable neurostimulator 128 to implantable neurostimulator 104, or vice versa. A pairing request may trigger a generation of a unique symmetric encryption key at both of implantable neurostimulator 104 and wearable neurostimulator 128. A unique symmetric encryption key may be generated by algorithms such as Elliptic Curve Diffie-Hellman (ECDH) key exchange protocols. Additional security mechanisms may take place in a pairing process between implantable neurostimulator 104 and wearable neurostimulator 128. For instance, a challenge-response protocol may be employed. A challenge-response protocol may be where either implantable neurostimulator 104 or wearable neurostimulator 128 sends a random challenge value and the other device must respond with a correct hash of the random challenge value. Mutual authentication may also be enforced by having both implantable neurostimulator 104 and wearable neurostimulator 128 authenticate each other using their unique credentials such as public/private key pairs, digital certifications, or device-specific passwords. Communication data may be encrypted with encryption algorithms such as, but not limited to, AES-256 or ChaCha20-Poly1305 AEAD. Encryption keys may be secretly stored in one or more dedicated cryptographic hardware modules of either or both of implantable neurostimulator 104 and wearable neurostimulator 128.

Dedicated cryptographic hardware modules may include, but are not limited to, Trusted Platform Modules (TPMs), secure enclaves, or other modules. Security protocols may be implemented in either or both of firmware and/or software of implantable neurostimulator 104 and wearable neurostimulator 128. In some embodiments, network 156 may include a secure communication channel between implantable neurostimulator 104 and wearable neurostimulator 128. A secure communication channel may utilize one or more protocols such as Transport Layer Security (TLS) or Bluetooth LE secure connections. Network 156, implantable neurostimulator 104, and/or wearable neurostimulator 128 may be i compliance with one or more security standards for medical devices, such as, but no limited to, IEC 62443, UL 2900, and/or FDA guidance. One or more external computing devices may provide implantable neurostimulator 104 and/or wearable neurostimulator 128 with firmware updates to proactively identify and mitigate potential security vulnerabilities.

Referring still to FIG. 1, first wireless communication device 116 may include one or more antennas. For instance, first wireless communication device 116 may include a trace antenna which may be printed on a printed circuit board (PCB). A PCB of implantable neurostimulator 104 may be a flexible PCB, in some embodiments. One or more components of implantable neurostimulator 104 may be disposed on a flexible PCB, which may allow implantable neurostimulator 104 to conform to a curvature of an inside of a patient’s body. First wireless communication device 116 may be placed close to a surface of a housing of implantable neurostimulator 104, which may allow for electromagnetic wave propagation through tissue of a patient. Second wireless communication device 140 may include one or more antennas. For instance, second wireless communication device 140 may include a monopole antenna, such as, but not limited to, a flexible helical monopole antenna. Second wireless communication device 140 may be configured to emit a radiation pattern from different on-body positions. Radiation patterns may include, but are not limited to, spherical hemispherical, or other radiation patterns. Second wireless communication device 140 may be positioned on a side of a housing of wearable neurostimulator 128. For instance, second wireless communication device 140 may be positioned to face towards implantable neurostimulator 104 when worn by a patient. An alignment of second wireless communication device 140 to first wireless communication device 116 may improve or maximize a line-of-sight communication between implantable neurostimulator 104 and wearable neurostimulator 128.

In some embodiments, a spatial delivery system may be implemented in either or both of first wireless communication device 116 and second wireless communication device 140. A “spatial diversity system” as used in this disclosure refers to an arrangement of multiple antennas. For instance, first wireless communication device 116 and/or second wireless communication device 140 may include two or more antennas. First processor 112 and/or second processor 144 may be configured to utilize a switching algorithm, which may select an antenna out of a plurality of antennas having a highest signal quality based on a received signal strength indicator (RRSI) measurement. Power combining techniques may additionally or alternatively be utilized by first processor 112 and/or second processor 144. Power combining techniques may include utilize signals from multiple antennas after weighting them based on individual signal strengths. In some embodiments, wearable neurostimulator 128 may be configured to utilize one or more beamforming techniques. Beamforming techniques may include, but are not limited to, electronically steerable passive array radiator (ESPAR) antennas. ESPAR antennas may focus radiation toward an implant location of implantable neurostimulator 104 which may enhance signal strength. Implantable neurostimulator 104 may employ backscatter modulation techniques, which may help optimize power efficiency. Out-of-band interference filtering techniques and/or strategic placement away from one or more noise sources may improve signal resilience of either or both of implantable neurostimulator 104 and wearable neurostimulator 128. Q factor components ma y utilized in one or more impedance matching circuits of implantable neurostimulator 104 and/or wearable neurostimulator 128, which mat help prevent interfere from moisture, temperature variations, and/or other factors.

With continued reference to FIG. 1, in some embodiments, first processor 112 and/or second processor 144 may be configured to perform one or more adaptive communication strategies. Adaptive communication strategies may include the use of, but not limited to, reinforcement learning, Bayesian optimization, and/or artificial neural networks (ANN). First processor 112 and/or second processor 144 may adjust transmission parameters such as, but not limited to, power, frequency, modulation scheme, and/or channel allocation base don real-time quality assessment of first link 160 and/or second link 164. Optimization techniques may be employed, such as, but not limited to, reinforcement learning. First processor 112 and/or second processor 144 may continuously monitor one or more performance metrics such as, but not limited to, Signal-to-Noise Ratio (SNR), Bit Error Rate (BER), Frame Error Rate (FER) and/or latency. As a non-limiting example, a Q-learning based algorithm may be implemented on first processor 112 and/or second processor 144 which may adapt transmission power and frequency of first link 160 and/or second link 164 based on observer BER. In some embodiments, first processor 112 and/or second processor 144 may utilize a machine learning model to determine optimal transmission parameters based on one or more factors. A machine learning model may be trained to input parameters such as SNR, BER, FER, and/or latency, and may output one or more transmission parameters, modulation schemes, and the like. Training data may be received via user input, external computing devices, and/or previous iterations of processing.

In some embodiments, transmission power of first link 160 may be adjusted by first processor 112 and/or transmission power of second link 164 may be adjusted by second processor 144. For instance, first link 160 and/or second link 164 may be adaptively adjusted between about 1mW to about 10mW, which may maintain a reliable link to network 156 while optimizing power consumption. A modulation scheme may first link 160 and/or second link 164 may be adaptively changed from BPSK to QPSK. A modulation scheme may be adaptively changed base don factors such as, but not limited to, link quality, noise resilience, data rate, and/or other factors. In some embodiments, first processor 112 and/or second processor 144 may employ one or more frequency hopping techniques. Frequency hopping techniques may span between about 400 MHz to about 2.4 GHz. Network 156 may include two or more communication channels, which first processor 112 may selectively switch first link 160 to and/or second processor 144 may selectively switch second link 164 to. Implantable neurostimulator 104 and/or wearable neurostimulator 128 may include one or more dedicated signal processors (DSPs) and/or field-programmable gate arrays (FPGAs) that may assist in real-time processing and low-latency performance of network 156. First processor 112 and/or second processor 144 may use one or more digital filters, matrix operations, and/or fast Fourier transforms for real-time analysis.

Still referring to FIG. 1, a direct communication between implantable neurostimulator 104 and wearable neurostimulator 128 may allow for an exchange of internal data 120, external data 136, and stimulation parameters of both internal stimulation output 124 and external stimulation output 152 between both devices. For instance, wearable neurostimulator 128 may receive internal data 120 and implantable neurostimulator 104 may receive external data 136. In some embodiments, based on external data 136, first processor 112 of implantable neurostimulator 104 may be configured to adjust one or more parameter settings of internal stimulation output 124. Likewise, second processor 144 may be configured to adjust one or more parameter settings of external stimulation output 152 based off internal data 120. Second processor 144 may adjust an intensity, frequency, duration, external target area, pulse width, and/or other parameters of external stimulation output 152 based on external data 136 and/or internal data 120. First processor 112 may adjust an intensity, frequency, duration, internal target area, pulse width, and/or other parameters of internal stimulation output 124 based on internal data 120 and/or external data 136. In some embodiments, first processor 112 may deactivate internal stimulator 122 for a period of time based on external data 136. Likewise, second processor 144 may deactivate external stimulator 148 based on internal data 120 for a period of time.

In some embodiments, outputs of internal stimulation output 124 and external stimulation output 152 may include one or more waveforms. Waveforms may include square waves. A square wave may have a pulse with of about 50 microseconds to about 450 microseconds with a frequency range of about 2Hz o about 200Hz. An amplitude of a square wave may be between about 0V to about 3V, about 1V to about 5V, about 2V to about 10V, or greater than 10V. Duty cycles may represent a percentage of time a square wave is at a high voltage level during one period, and ma be varied between about 10% to about 90%, greater than 90%, or less than 10%. In some embodiments, waveforms may include burst waveforms, such as a BurstDR stimulation waveform. A burst waveform may deliver sequences of pulses in burst, wherein each burst may include a number of spikes in amplitude with a configurable intra-burst frequency. A number of spikes in amplitude may range from about 1 spike to about 10 spikes per burst, in some embodiments. An intra-burst frequency may range from about 200Hz to about 500Hz, less than about 200Hz, or greater than about 500Hz. An inter-burst frequency of a burst waveform may be adjustable between about 10Hz to about 100Hz, less than about 10Hz, or greater than about 100Hz. Pulses within a burst waveform may have a pulse width of about 0.5 milliseconds to about 2 milliseconds, less than about 0.5 milliseconds, or greater than about 2 milliseconds. An amplitude of a burst waveform may be between about 1V to about 10V, less than about 1V, or greater than about 10V. As a non-limiting example, a burst waveform of 0.75 ms pulse width and 4V amplitude could be effective for an essential tremor application, while a 1.5 ms pulse width and 8V amplitude may be suited for a Parkinson's disease application. One or more DACs and/or amplifiers may be employed to boost a burst waveform signal.

In some embodiments, outputs of internal stimulation output 124 and external stimulation output 152 may include high frequency outputs. High frequency outputs may include waveform frequencies of about 130Hz to about 1KHz, less than about 130Hz, or greater than about 1KHz. Beta band stimulation may modulates neural circuits involved in motor control, providing a mechanism to manage symptoms of movement disorders. For example and without limitation, stimulating the subthalamic nucleus or globus pallidus interna in the beta band range could help alleviate motor dysfunction in Parkinson’s disease in a patient. A frequency of about 13Hz to about 30Hz may be adjusted based on a neural biomarker feedback, such as local field potential power received from internal data 120.

In some embodiments, outputs of internal stimulation output 124 and external stimulation output 152 may include beta band stimulation. Beta band stimulation may have a frequency band ranging from about 13Hz to about 30Hz, which may be associated with motor control and may be linked to symptoms of movement disorder such as Parkinson’s disease and essential tremor.

In some embodiments, outputs of internal stimulation output 124 and external stimulation output 152 may include Gamma band stimulation. Gamma band stimulation may include a frequency of about 0MHz to about 12MHz. In some embodiments, Gamma band stimulation may include a frequency of about 30Hz to about 80Hz. As a non-limiting example, first processor 112 may measure accelerometer data indicative of tremor severity, and when the amplitude of tremor movement exceeds a predefined threshold, first processor 112 may increase the gamma band stimulation amplitude by 2V and decrease the frequency by 5Hz to better disrupt the underlying pathological neural activity. Stimulation parameters may be continually adjusted.

In some embodiments, a direct communication between implantable neurostimulator 104 and wearable neurostimulator 128 may form a closed-loop system between both devices. A closed-loop system of implantable neurostimulator 104 and wearable neurostimulator 128 may include each device providing stimulation output and adjusting the stimulation output according to input data 120 and external data 136 without additional input. In some embodiments, implantable neurostimulator 104 may act in a closed-loop system separate from wearable neurostimulator 128, which may additionally or alternatively act in a closed loop system. Closed loop systems may include the use of proportional integral derivative (PID) algorithms. A PID algorithm may have a proportional (“P”) term, and integral (“I”) term, and/or a derivative (“D”) term. A PID algorithm may be modified for use in neurostimulation by first processor 112 and/or second processor 144. For instance, a P term may be a value directly proportional to a current error. A current error may be a difference between a desired or ideal value and an actual or measured value. As a non-limiting example, a P value may be an calculated error value between a measured tremor intensity and a desired tremor intensity. IN some embodiments, a proportional response of a PID algorithm may by tuned by one or more gain factors. A gain factor may represent a multiplication value that may increase a value of a P term. A gain factor may be less than about 1 or greater than about 1, without limitation. In some embodiments, an I term may represent an accumulation of past errors over time. Errors may be caused by sensor drift, changing in baseline neurological states, and/or other factors. An I term may compensate for sensor drift, changes in baseline neurological states, and/or other factors by adjusting one or more stimulation parameters to maintain a desired neurological state. An I term may have one or more gain factors that may be multiplication values of less than about 1 or greater than about 1, without limitation. A D term may represent a rate of change in an error and may be calculated to anticipate future trends based on current system inputs and/or outputs. For instance, and without limitation, if error within a system is rapidly increasing, a D term may ramp up one or more stimulation parameters in an anticipatory manner to mitigate potentially worsening symptoms of a patient. A strength of a D term may be tuned by one or more gain factors, which may be multiplication values of less than about 1 or greater than about 1, without limitation.

In some embodiments, a P term, I term, and/or D term may be amalgamated using a weighted sum to generate one or more control signals that may module stimulation parameters of internal stimulation output 124 and/or external stimulation output 152. As a non-limiting example, in an essential tremor application, a PID controller may increase a stimulation amplitude and/or frequency proportional to a measured tremor intensity to suppress the tremor. As another non-limiting example, in a Parkinson’s disease application, a PID algorithm may module a stimulation pulse width to maintain a desired beta band power level indicative of optimal motor control. A PID control loop may run continuously with internal data 120 and/or external data 136 fed back to first processor 112 and/or second processor 144.

First processor 112 and/or second processor 144 may implement an adaptive control technique, in which real-time changes in internal data 120 and/or external data 136 may cause first processor 112 and/or second processor 144 to adjust one or more parameter settings of internal stimulation output 124 and/or external stimulation output 152. Adaptive control techniques may include, but are not limited to, nonlinear autoregressive exogenous (NARX) neural network models. NARX models may be trained on historical input-output data to learn a dynamic relationship between stimulation parameters such as, but not limited to, amplitude, pulse width, and/or frequency and neurological measurements such as, but not limited to, limb accelerations and/or local field potentials. Adaptive control techniques may include linear state space models that may be estimated using subspace identification and/or Kalman filter algorithms, which may represent an evolution of neurological states in response to stimulation inputs. In some embodiments, model reference adaptive control (MRAC) may be utilized. In an MRAC architecture, one or more parameters may be tuned to minimize an error between a model-predicted output and an actual measured output. The MIT rule, Lyapunov theory-based update laws, and/or gradient descent may be used to update gain and/or weight values to track a reference model. In some embodiments, a Self-Tuning Regulator (STR) may be implemented using a recursive prediction error or least squares identification method to re-estimate model parameters based on new input-output data and may update one or more control laws accordingly. In some embodiments, an extremum seeking control algorithm may be used. An extremum seeking control algorithm may optimize an objective function in real-time by continuously adjusting parameters an observing neurological responses.

With continued reference to FIG. 1, first processor 112 and/or second processor 144 may be configured to utilize a model predictive control (MPC) algorithm. An MPC algorithm may predict future behavior of a neurological state in response to various possible stimulation parameter settings. A cost function in an MPC algorithm may be defined to quantitatively assess desirability of predicted future sates, and an optimization of the cost function may be carried out to determine one or more optimal stimulation parameters. A cost function may include terms reflecting deviation from a desired neurological state, energy consumption of implantable neurostimulator 104 and/or wearable neurostimulator 128, and/or possible discomfort or side effects associated with stimulation. As a non-limiting example, a cost function may include a weighted term for an error between predicted and desired tremor intensity based on accelerometer data to minimize tremor symptoms, a weighted term representing power consumption to maximize battery life, and/or a term reflecting subjective patient feedback on stimulation comfort to minimize side effects. Relative weightings of one or more terms may be optimized based on clinical needs, patient data, and/or other factors. An MPC algorithm may be utilized by first processor 112 and/or second processor 144 to predict future tremor intensity based on a correlation between parameters of internal stimulation output 124 and/or external stimulation output 152 to tremor levels derived from internal data 120 and/or external data 136. In some embodiments, an autoregressive exogenous (ARX) model structure may be employed. An ARX model structure may represent a relationship between one or more parameters of internal stimulation output 124 and/or external stimulation output 152 to measured tremor intensity based on past values of internal stimulation output 124 and/or external stimulation output 152 and/or internal data 120 and/or external data 136. An ARX model may use a cost function, which may include a quadratic cost function, to penalize large deviations from a desired level of tremor suppressions. A cost function may include one or more linear terms representing stimulation power consumption which may be used to minimize batter usage. A sequential quadratic programming (SQP) algorithm may be utilized to solve optimization problems with linear and/or nonlinear constrains one or more stimulation parameters of internal stimulation output 124 and/or external stimulation output 152. Recursive least squares and/or Bayesian estimation techniques may be used to adapt parameters of internal stimulation output 124 and/or external stimulation output 152.

In some embodiments, implantable neurostimulator 104 and wearable neurostimulator 128 may operate in a sequential processing architecture. A “sequential processing architecture” as used herein refers to an arrangement of devices that operate in an order. For instance, implantable neurostimulator 104 may be a primary neurostimulator and wearable neurostimulator 128 may be a secondary or auxiliary device, or vice versa. A primary device may receive data from a secondary device over a low-latency wireless communication link, such as network 156, and may adjust one or more stimulation output parameters based on the received data. A primary device may process received data along with its own sensed data. A primary device may adjust parameters of stimulation output of a secondary device and/or of its own stimulation output, in some embodiments. For instance, a primary device may receive sensed data from a secondary device and may adjust one or more stimulation parameters of a stimulation output of the secondary device and/or its own stimulation output.

Referring still to FIG. 1, first processor 112 and/or second processor 144 may be configured to utilize one or more Kalman filters. For instance, first processor 112 and/or second processor 144 may utilize a multi-input multi-output (MIMO) state space model relating one or more parameters of internal stimulation output 124 and/or external stimulation output 152 as input to one or more neural signals from local field potentials or electromyography as outputs. Local field potentials and/or electromyography may be obtained from internal data 120 and/or external data 136. A MIMO model may include non-linear elements which may be used to represent action potential generation. A Kalman filter algorithm may be used in conjunction with a MIMO model to estimate current states of a neurological system of a patient. In some embodiments, implantable neurostimulator 104 and/or wearable neurostimulator 128 may include a dedicated Kalman filter hardware accelerator.

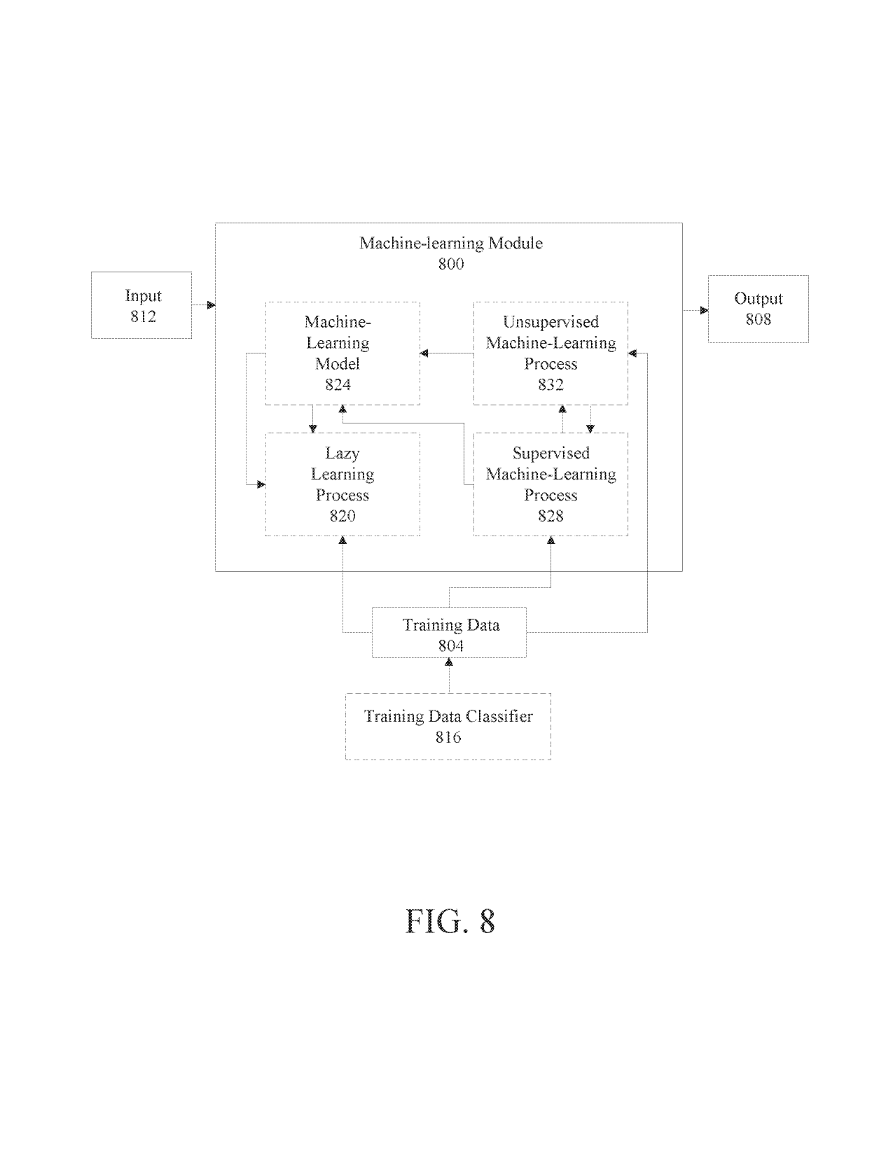

In some embodiments, first processor 112 and/or second processor 144 may be configured to utilize one or more machine learning models and/or neural networks. Machine learning models may include, but are not limited to, supervised learning models, unsupervised learning models, reinforcement learning models, deep Q-learning models, and/or other models. Neural networks may include recurrent neural networks, long short-term memory networks, hybrid approaches, and/or other types of neural networks. Inputs to one or more machine learning models and/or neural networks may include amplitudes, pulse widths, frequencies, waveform shapes, internal stimulator 122 selection, external stimulator 148 selection, internal data 120, and/or external data 136. Outputs of one or more neural networks and/or machine learning models may include one or more parameters of internal stimulation output 124 and/or external stimulation output 152. In some embodiments, one or more machine learning models may be run locally on first processor 112 and/or second processor 144. In other embodiments, one or more machine learning models may operate on an external computing device and may communicate data to first processor 112 through first wireless communication device 116 and/or second processor 144 through second wireless communication device 140. In some embodiments, one or more machine learning models may be trained on one or more external computing devices and parameters and/or weights of the trained one or more machine learning models may be communicated to first processor 112 through first wireless communication device 116 and/or second processor 144 through second wireless communication device 140. Machine learning models may be described in further detail below with reference to FIG. 8.

First processor 112 and/or second processor 144 may additionally or alternatively be configured to utilize one or more optimization algorithms. Optimization algorithms may include, but are not limited to, linear programming, quadratic programming, evolutionary algorithms, and/or other algorithms. An optimization algorithm may aim to find one or more parameters of internal stimulation output 124 and/or external stimulation output 152 that may minimize or maximize a defined objective function. An objective function may relate to symptoms of movement disorders, neurological disorders, and/or other disorders. An objective function may include one or more terms reflecting deviations from a desired neurological state, energy consumption, and/or discomfort associated with stimulation.

In some embodiments, first processor 112 and/or second processor 144 may be configured to utilize fuzzy logic control (FLC). An FLC system may include one or more inference rules, fuzzification of values, defuzzification of values, and/or other types of FLC parameters. FLC may convert binary inputs into fuzzy values using a membership function. In some embodiments, an FLC system may include one or more inferences that may apply one or more sets of fuzzy rules to determine one or more output fuzzy sets. For instance and without limitation, "Tremor_Severity" could be classified as "Low", "Medium", or "High" based on accelerometer data. A stimulation amplitude of internal stimulation output 124 and/or external stimulation output 152 may be represented by fuzzy sets like "Low_Amp", "Medium_Amp", and "High_Amp". Triangle or trapezoid membership functions may map input values to corresponding fuzzy sets. A rule base may be defined. A rule base may include a number of IF-THEN rules linking linguistic variables of input and output, e.g. "IF Tremor_Severity is High THEN Stim_Amplitude is Low_Amp. An FLC system may include an implication method that may determine fuzzy rule outputs, an aggregation that may combine outputs, and a defuzzification that may convert fuzzy outputs into one or more parameters of internal stimulation output 124 and/or external stimulation output 152. An FLC system may include a type-2 FLC system, in some embodiments.

Still referring to FIG. 1, in some embodiments, first processor 112 and/or second processor 144 may be configured to utilize one or more genetic algorithms (GA). Genetic algorithms may be optimization algorithms based on the process of natural selection. Gor instance, GA systems may operate through iterative processes of selection, crossover, and/or mutation to create a multi-dimensional parameter space and discover optimal or near-optimal solutions that may maximize or minimize a defined objection function. For instance, a GA system may search through a parameter space including a plurality of parameters of internal stimulation output 124 and/or external stimulation output 152. Parameters may include, but are not limited to, amplitudes of about 1V to about 10V, frequencies of about 130Hz to about 185Hz, pulse widths of about 60 μs to about 200 μs, and/or other parameters. In some embodiments, a GA system may encode one or more parameters to be optimized as chromosomes, with each parameter value encoded as a gene. For instance and without limitation, amplitude, frequency, and pulse width could be encoded as genes in a chromosome. An initial population of stimulation parameter combinations may be generated, either randomly or seeded based on clinical data. A fitness of each chromosome may be evaluated by applying stimulation parameters in simulation or to a patient, and measuring a resulting objective function value. Selection operators like roulette wheel or tournament selection may be applied to bias reproduction towards fitter patients. Crossover operators may combine genes from pairs of selected parents to breed new solutions. For instance and without limitation, single-point crossover may combine amplitude and frequency genes from one parent, with pulse width genes from the other. Mutation operators such as bit flipping may be used to introduce randomness, which may help avoid local optima. An evaluation-selection-crossover-mutation cycle of a GA system may be repeated over generations until convergence criteria are met, such as parameter variance under a threshold or maximum generations reached. An optimal parameter combination may be applied to modulate outputs of stimulation output 124 and/or external stimulation output 152.

In some embodiments, first processor 112 and/or second processor 144 may be configured to utilize Bayesian optimization. Bayesian optimization may include constructing a probabilistic surrogate model of an objective function to guide a search through a parameter space. For instance, Bayesian optimization may include building a Gaussian Process surrogate model, which may be a probabilistic non-parametric model that may provide uncertainty estimates. A Gaussian process may be initialized with a small set of randomly sampled points or based on prior knowledge. An acquisition function may be formulated to determine e point to evaluate, such as an Upper Confidence Bound criteria which may trade off exploration and exploitation. An acquisition function may be maximized using an optimization algorithm, such as L-BFGS-B, to find a parameter settings to test. An objective function may be a deviation from a desired tremor level based on accelerometer data, in some embodiments. A Gaussian Process model may be updated to incorporate one or more new data values. An iterative process of Bayesian inference and/or acquisition maximization may progressively refine a surrogate model and may zone in on optimal parameters. One or more parameters optimized by a Bayesian process may be deployed in outputs of stimulation output 124 and/or external stimulation output 152.

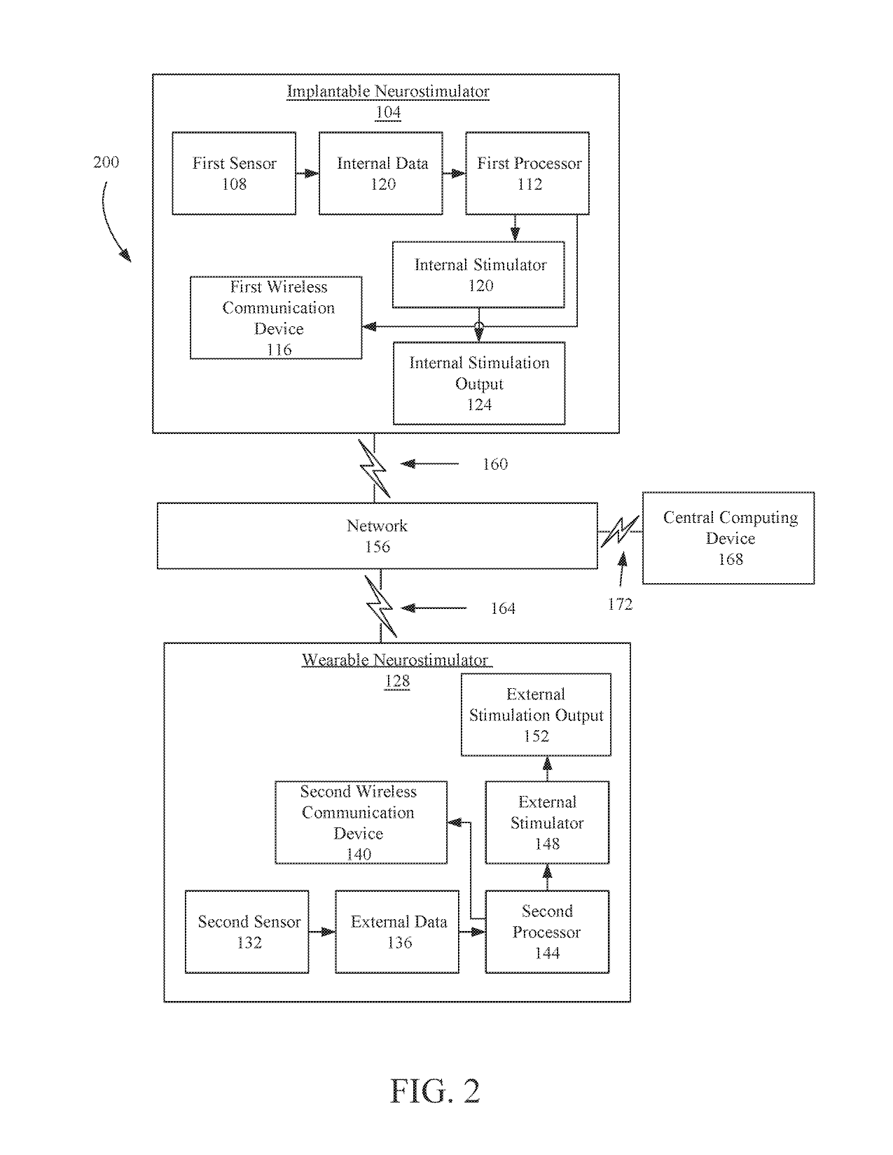

Referring now to FIG. 2, the neurostimulation system 100 of FIG. 1 within a centralized processing architecture 200 is presented. Architecture 200 may include central computing device 168. Central computing device 168 may be a smartphone, laptop, desktop, server, or any other computing device. Central computing device 168 may communicate with network 156 via third link 172. Third link 172 may be a Wi-Fi, Bluetooth, Cellular, or other link. Central computing device 168 may communicate data to implantable neurostimulator 104 and/or wearable neurostimulator 128 via network 156. For instance, internal data 120, parameters of internal stimulation output 124, metrics of internal stimulator 120, external data 136, parameters of external stimulation output 152, and/or metrics of external stimulator 148 may be communicated with central computing device 168 via network 156. Central computing device 168 may be configured to determine parameters of internal stimulation output 124 and/or external stimulation output 152 based on internal data 120 and/or external data 136. Calculation of parameters of simulation outputs 124 and 152 may be offloaded to computing device 164, which may reduce computational power requirements of first processor 112 and/or second processor 144. In some embodiments, central computing device 168 may be configured to utilize one or more machine learning models. A machine learning model may be trained with training data correlating internal data 120 and/or external data 136 to parameters of stimulation outputs 124 and/or 152. Training data may be received via user input, external computing devices, and/or previous iterations of processing. In some embodiments, central computing device 168 may be configured to use a machine learning model to calculate one or more parameters of stimulation outputs 124 and/or 152. Central computing device 168 may communicate one or more parameters of stimulation outputs to implantable neurostimulator 104 and/or wearable neurostimulator 128 via network 156 through third link 172. For instance, network 156 may include implantable neurostimulator 104, wearable neurostimulator 128, and central computing device 168, each of which may be in direct or indirect communication with one another.

In some embodiments, central computing device 168 may be configured to optimize communication of data of network 156 through a machine learning model. For instance, a machine learning model may be trained with training data correlating data of first link 160 and/or second link 164, data of network 156, and/or data of third link 172 to one or more transmission parameters. Training data may be received via user input, external computing devices, and/or previous iterations of processing. Central computing device 168 may be configured to determine transmission parameters of first link 160, second link 164, and/or third link 172 through a machine learning model. In some embodiments, central computing device 168 may be configured to utilize any optimization and/or learning algorithm described herein. Central computing device 168 may be configured to perform any networking or encryption process described herein. For instance, central computing device 168 may encrypt network 156 and/or third link 172 and/or may adjust any transmission parameters of first link 160, second link 164, and/or third link 172 using any networking and/or optimization technique described herein.

Central computing device 168 may be configured to receive internal data 120 and/or external data 136 and calculate one or more parameters of internal stimulation output 124 and/or external stimulation output 152. In some embodiments, central computing device 168 may be configured to determine a neurological state of a patient based on internal data 120 and/or external data 136. A neurological state may include severity of movement disorders, severity of mental disorders, and/or other severities of other disorders. In some embodiments, central computing device 168 may be configured to utilize a classifier or other machine learning model to determine a neurological state of a patient. A classifier may be trained with training data correlating internal data 120 and/or external data 136 to categories of neurological states. Categories of neurological states may include, but are not limited to, low severity movement disorder symptoms, medium severity movement disorder symptoms, high severity movement disorder symptoms. In some embodiments, categories of neurological states may include levels of depression, obsessive compulsive disorder (OCD), and/or other neurological disorders. Training data may be received via user input, external computing devices, and/or previous iterations of processing.

Central computing device 168 may input internal data 120 and/or external data 136 into a classifier, which may output a neurological state of a patient. In some embodiments, central computing device 168 may be configured to calculate one or more parameters of stimulation outputs 124, 152, based on a neurological state of a patient. For instance, if a neurological state of a patient is indicative of low levels of movement disorder symptoms, central computing device 168 may lower frequencies, amplitudes, and/or other parameters of outputs 124, 152. In some embodiments, if a neurological state of a patient indicates high levels of movement disorder symptoms, central computing device 168 may increase frequencies, amplitudes, and/or other parameters of outputs 124, 152. In some embodiments, central computing device 168 may be configured to increase one or more parameters of internal stimulation output 124 while decreasing one or more parameters of external stimulation output 152, and/or vice versa. In some embodiments, central computing device 168 may completely deactivate implantable neurostimulator 104 and/or wearable neurostimulator 128 based on internal data 120 and/or external data 136.

In some embodiments, system 200 may operate in a hybrid architecture. A “hybrid architecture” as used in this disclosure refers to a combination of direct and central architecture. In a hybrid architecture, implantable neurostimulator 104 and wearable neurostimulator 128 may communicate directly in addition to communication with central computing device 168. Implantable neurostimulator 104 and wearable neurostimulator 128 may communicate internal data 120 and/or external data 136 with each other and may adjust their respective outputs accordingly. In parallel, central computing device 168 may be in communication with either or both of implantable neurostimulator 104 and wearable neurostimulator 128 and may generate additional calculations such as, but not limited to, networking optimizations, predictions of future neurological states, battery optimization, user discomfort levels, and/or other calculations.

Still referring to FIG. 2, in some embodiments, central computing device 168 may be in communication with one or more user interfaces (UIs). A patient may adjust any settings of implantable neurostimulator 104 and/or wearable neurostimulator 128 described herein via a UI of central computing device 168. In some embodiments, central computing device 168 may be configured to communicate data to a medical network and/or medical provider. For instance and without limitation, a medical network and/or medical provider device may communicate with central computing device 168 via a fourth link, which may allow the medical network and/or medical provider device to partially or completely access data of network 156, implantable neurostimulator 104, and/or wearable neurostimulator 128.

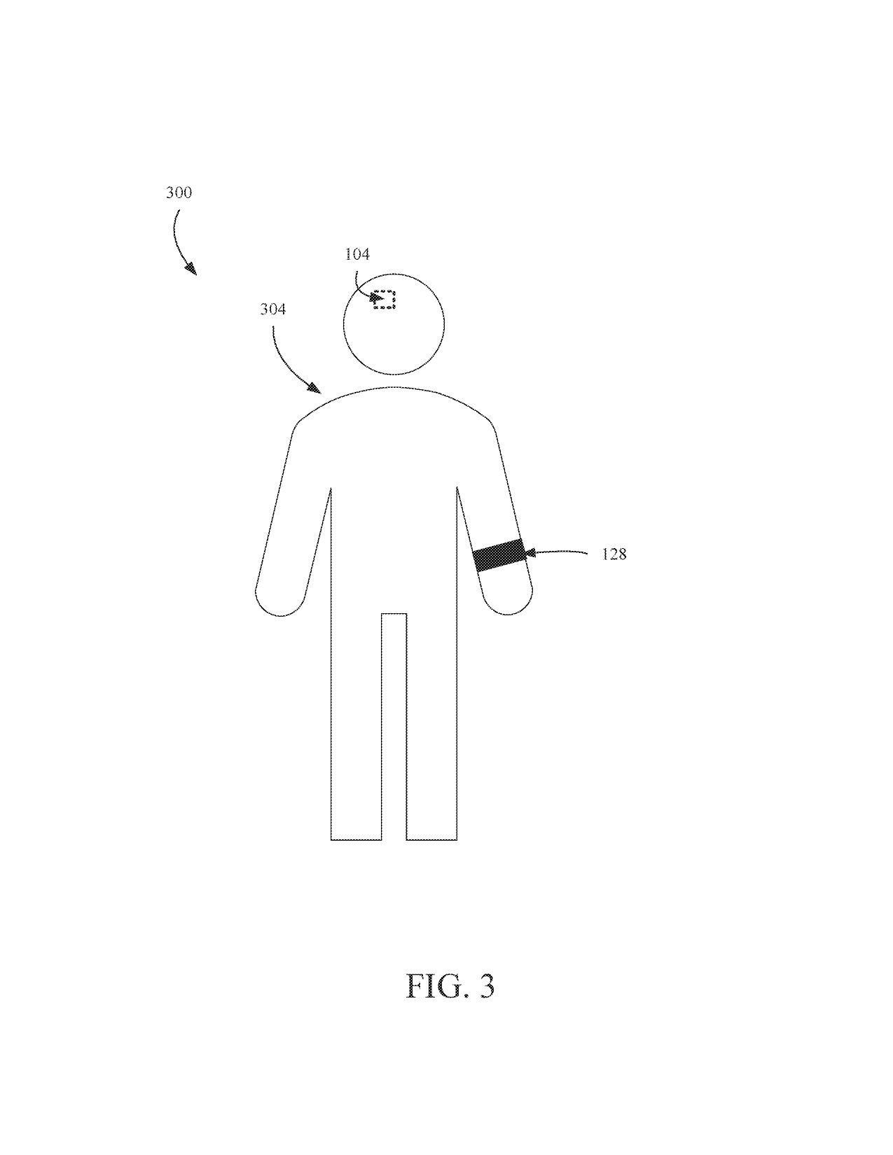

Referring now to FIG. 3, an illustration of a patient 304 using implantable neurostimulator 104 and wearable neurostimulator 128 is presented. Patient 304 may have one or more neurological disorders, such as, but not limited to, tremors, Parkinson’s, OCD, depression, anxiety, Dystonia, Epilepsy, Chronic Pain, and/or any other neurological disorder. Although depicted as a brain implant in FIG. 3 for illustrative purposes, this is solely for illustrative purposes and implantable neurostimulator 104 may be any type of implantable neurostimulation device, such as, but not limited to, brain implants, chest implants, spinal implants, and/or other implants. Although depicted as a wrist-band device in FIG. 3, this is only for illustrative purposes and wearable neurostimulator 128 may be any type of wearable device, such as, but not limited to, headbands, helmets, vest, shirts, gloves, ankle or foot-worn devices, patch devices, belt devices, and/or any other device.

In some embodiments, implantable neurostimulator 104 may be configured to sense internal data and generate an internal stimulation output, as described above with reference to FIGS. 1-2, without limitation. Wearable neurostimulator 128 may be configured to sense external data and generate an external stimulation output, as described above with reference to FIGS. 1-2, without limitation. Implantable neurostimulator 104 may be in direct communication with wearable neurostimulator 128 via one or more links, networks, or other communication channels. In some embodiments, implantable neurostimulator 104 and wearable neurostimulator 128 may work in a sequential architecture. For instance, implantable neurostimulator 104 may be a primary device and wearable neurostimulator 128 may be a secondary or auxiliary device. Implantable neurostimulator 104 may receive data from wearable neurostimulator 128 and may adjust stimulation outputs of itself based on data received from wearable neurostimulator 128. In some embodiments, wearable neurostimulator 128 may be a primary device and implantable neurostimulator 104 may be a secondary device. In some embodiments, implantable neurostimulator 104 and wearable neurostimulator 128 may be in communication with an external computing device, such as, but not limited to, a smartphone, laptop, desktop, server, or other device.

Patient 304 may adjust one or more parameters of stimulation output through a UI of wearable neurostimulator 128. For instance, patient 304 may adjust network architectures such as direct architectures, sequential architectures, hybrid architectures, or any other architecture described herein. In some embodiments, patient 304 may remove wearable neurostimulator 128 from a body part of themselves, which may be communicated to implantable neurostimulator 104. For instance, wearable neurostimulator 128 may include one or more proximity sensors that may be configured to detect if wearable neurostimulator 128 is being worn. Implantable neurostimulator 104 may be configured to receive data indicating wearable neurostimulator 128 has been removed from patient 304 and may alter one or more parameters of a simulation output to compensate for the removal of wearable neurostimulator 128. In some embodiments, wearable neurostimulator 128 may be configured to notify patient 304 that a connection to implantable neurostimulator 104 has been made or has been lost and/or is weak. Notification may occur through one or more speakers and/or haptic vibrators of wearable neurostimulator 128. In some embodiments, wearable neurostimulator 128 and/or implantable neurostimulator 104 may be configured to communicate with a smartphone or web application. A smartphone or web application may be operable to run on a patient’s device, which may be a smartphone, laptop, desktop, tablet, or other device. A smartphone or web application may communicate data to patient 304 such as, but not limited to, calculated neurological states, projected neurological states, stimulation parameters, internal data, external data, recommended actions, and/or other information. In some embodiments, a smartphone or web application may provide guidance on positioning of wearable neurostimulator 128 to facilitate a strong connection to implantable neurostimulator 104. For instance, a smartphone or web application may show an illustration of a hand position for patient 304 to put their hand in to establish an initial connection between wearable neurostimulator 128 and implantable neurostimulator 104. In some embodiments, a smartphone or web application my provide an illustration to patient 304 of a correct or ideal positioning of wearable neurostimulator 128 and may notify patient 304 if wearable neurostimulator 128 is detected as being incorrectly positioned.

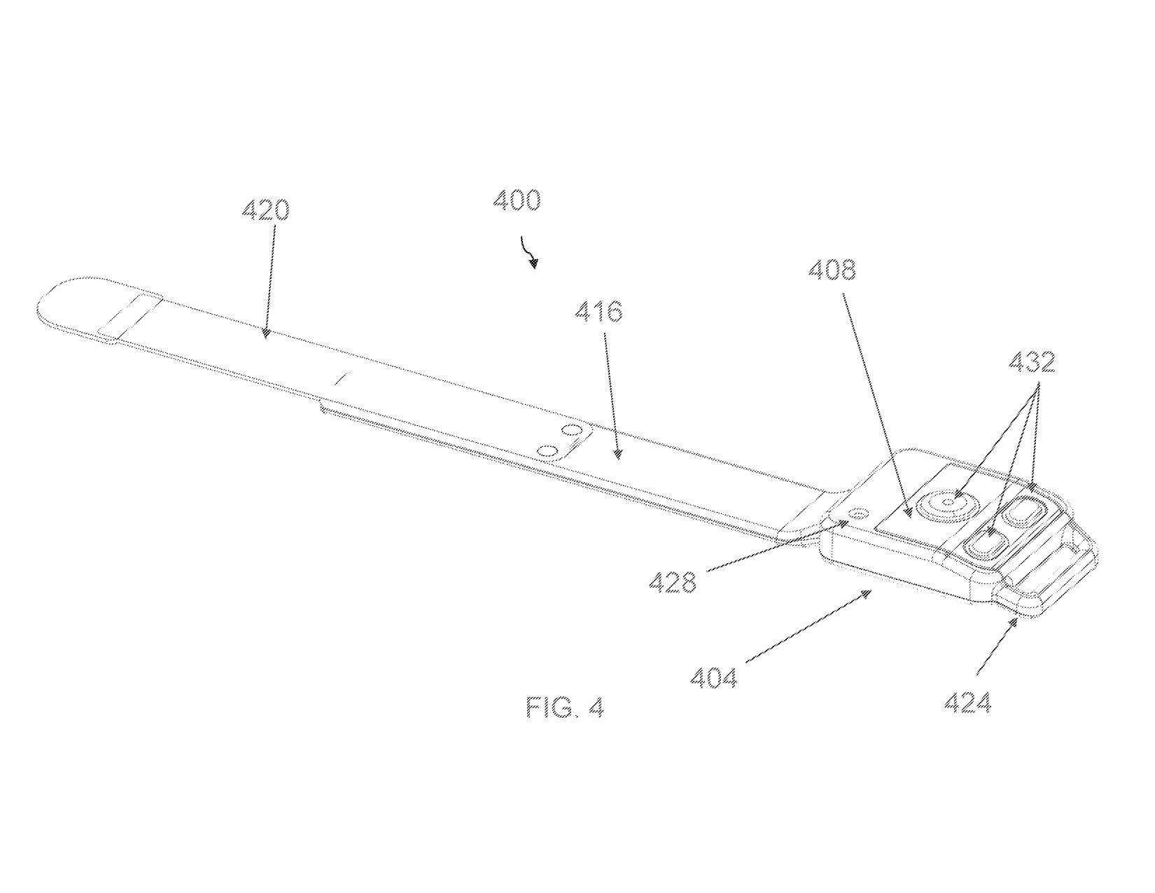

Referring now to FIG. 4, an illustration of a wearable neurostimulator 400 is presented. Wearable neurostimulator 400 (also referred to as a “wearable device”) 400 may include housing 404. Housing 404 may be rectangular, circular, or other shapes. In some embodiments, housing 404 may be designed to house control module 408. Control module 408 may include a controller, processor, or other device. Control module 408 may include one or more resistors, transistors, capacitors, sensors, or other electrical components. Control module 408 may have one or more interactive elements 432. Interactive elements 432 may include, but are not limited to, buttons, touch sensors, capacitive sensors, and/or other devices. Interaction of one or more interactive elements 432 may cause a processor of control module 408 to perform various functions. In some embodiments, interactive elements 432 may include a power button and two or more stimulation adjusting buttons. A power button may turn on an off wearable device 400, enable a pairing mode of wearable device 400, or perform other functions. Stimulator adjusting buttons of interactive elements 432 may adjust one or more parameters of a stimulation output generated by control module 408. In some embodiments, interactive elements 432 may include a first stimulation adjuster button and a second stimulation adjuster button. A first stimulation adjuster button may be configured to increase one or more parameters of a stimulation output while a second adjuster button may be configured to decrease one or more parameters of a stimulation output. Stimulator adjuster buttons of interactive elements 432 may adjust any parameter of a stimulation output described throughout this disclosure, without limitation.

Control module 408 may be removably couplable to housing 404 and/or wristband 416. For instance, housing 404 may be designed as a snap-in case, which may allow an insertion of control module 408 into housing 404 via a snaping mechanism. Wristband 416 may extend away from housing 404 and may be designed to wrap around a portion of a user’s body, such as a wrist or arm, without limitation. Wristband 416 may secure around a portion of a user’s body via securing element 420. Securing element 420 may be a hook and loop fastener, a magnetic strap, Velcro, and/or other securing devices. Wristband 416 may secure itself to slot 424 of housing 404. Slot 424 may be shaped to allow a width of wristband 416 to pass through itself. In some embodiments, a user may adjust a tension of wristband 416 by adjusting an amount of length of wristband 416 passing through slot 424. As a non-limiting example, a user may wrap wristband 416 around their wrist and insert an end of wristband 416 into slot 424. A user may tension wristband 416 through slot 404 and secure wristband 416 to itself via securing element 420. Housing 404 may include light emitting diode (LED) 428. LED 428 may be placed on a top facing surface of housing 404. In some embodiments, LED 428 may be placed at a top left or top right side facing surface of housing 404. LED 428 may emit one or more wavelengths of light, which may indicate various information to a user. LED 428 may be configured to emit pulses of light.

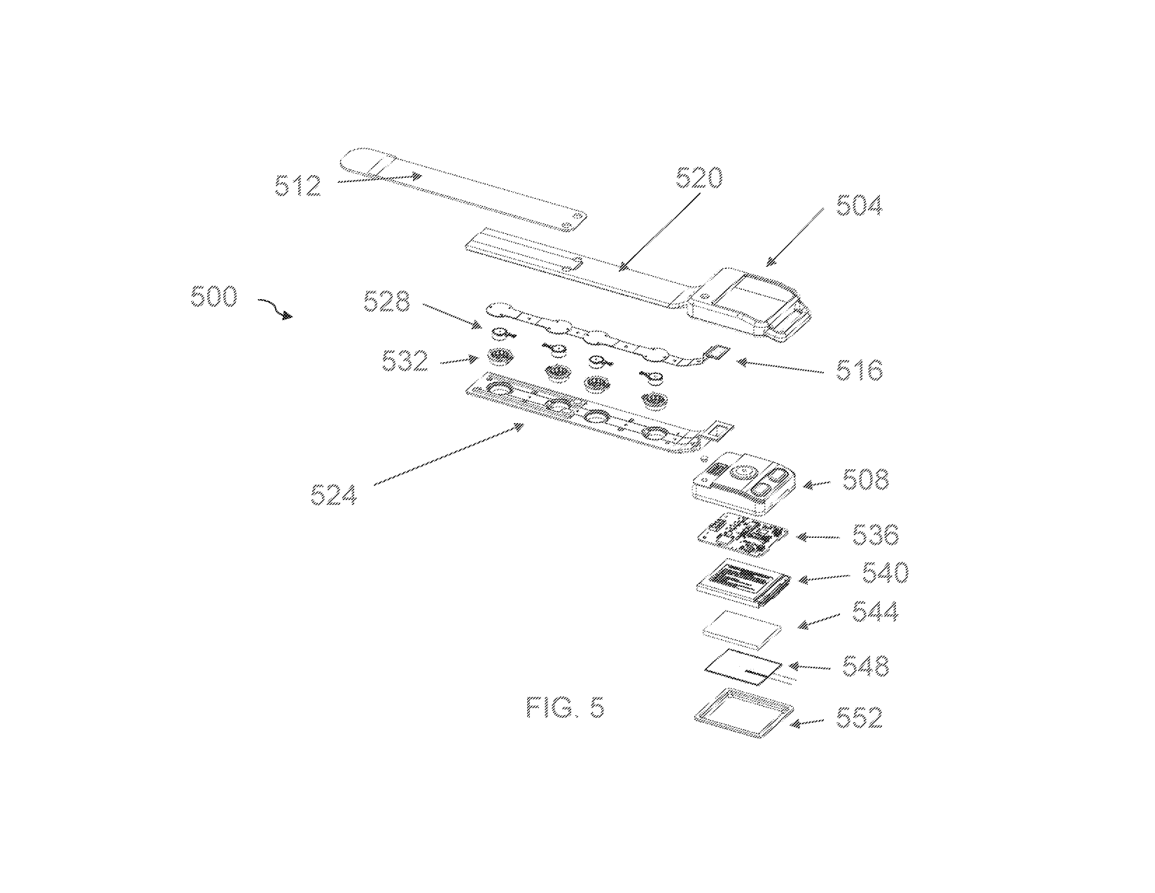

Referring now to FIG. 5, an exploded view of the wearable device of FIG. 4 is presented. Wearable device 500 may include housing 504, securing element 512, and control module 508, each of which may be as described above with reference to FIG. 4. Wearable device Wristband 500 may include a flexible printed circuit board (PCB) 516. Flexible PCB 516 may be positioned between top wristband half 520 and bottom wristband half 524. Flexible PCB 516 may be in electrical and/or mechanical communication with stimulators 528. Stimulators 528 may be, but are not limited to, electric, ultrasonic, heat, or vibratory. Wearable device 500 may include one or more motor caps 532, in embodiments where stimulators 528 may be vibratory. Control module 508 may include a main PCB 536, battery 540, foam spacer 544, charging coil 548, and/or a bottom housing component 552. Main PCB 536 may include processors, controllers, sensors, and/or other components. Main PCB 536 may be configured to connect to flexible PCB 516 via an electrical connection. Main PCB 536 may control one or more stimulators 528 via an electrical connection to flexible PCB 516. Battery 540 may be any type of battery, such as, but not limited to, lithium-ion, alkaline, or other batteries. Battery 540 may be configured to power main PCB 536, flexible PCB 516, stimulators 528, and/or other components. Battery 540 may be rechargeable via charging coil 548. Charging coil 548 may be configured to receive electrical power via electromagnetic induction. Charging coil 548 may provide power received via electromagnetic induction to battery 540. Foam spacer 544 may be positioned between battery 540 and charging coil 548, which may provide insulation to battery 540 from charging coil 548. Bottom housing component 552 may connect to a top housing component of control module 508. For instance, bottom housing component 552 may be placed underneath one or more components of control module 508 and may secure one or more components within an internal formed by a connection of bottom housing component 552 to a top housing component of control module 508.

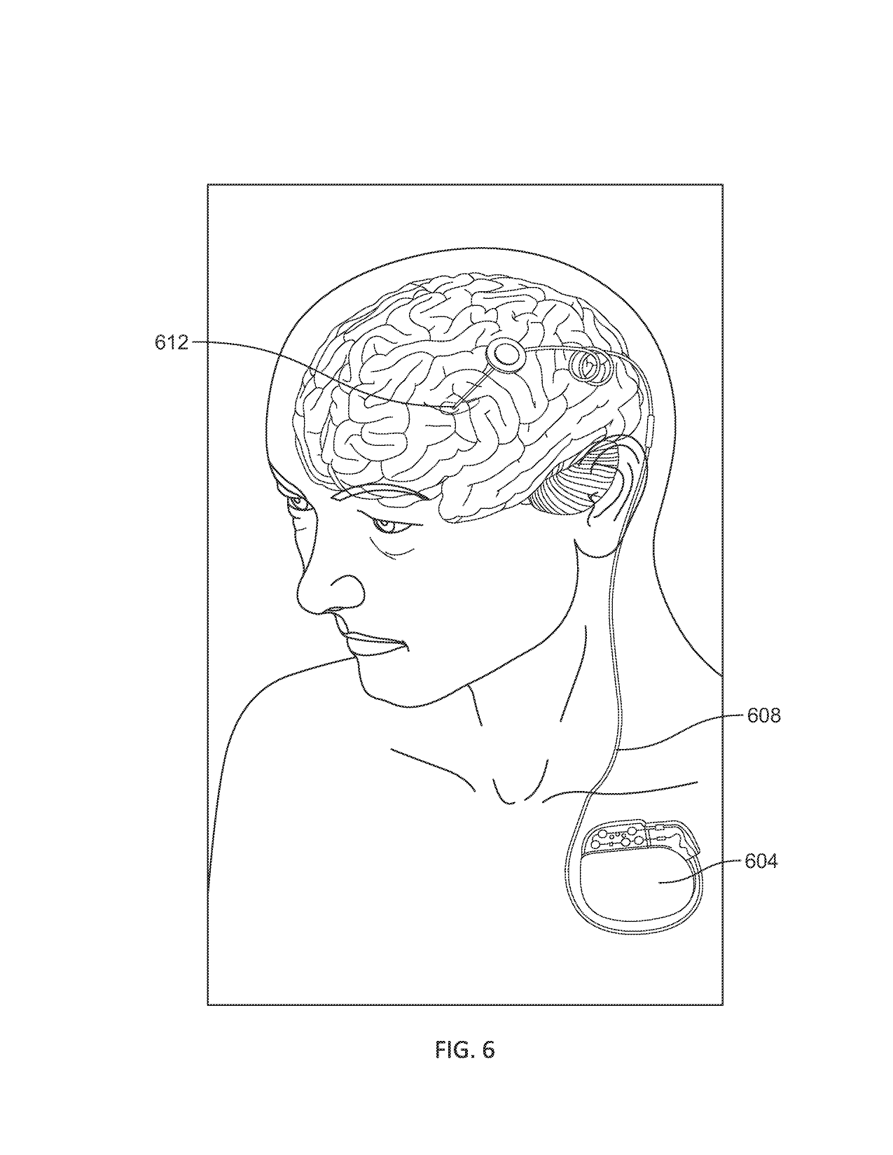

Referring now to FIG. 6, an embodiment of an implantable neurostimulator 600 is shown. Implantable neurostimulator 600 may be the same as described above with reference to FIGS. 1-2, without limitation. Although depicted as a chest implant, implantable neurostimulator 600 may be any type of implantable device as described herein, without limitation. Implantable neurostimulator 600 may be inserted into a chest of a patient and may connect to a brain of a patient through one or more wires 608. In some embodiments, implantable neurostimulator 604 may be inserted directly into a skull of a patient. Implantable neurostimulator 604 may be a DBS system, in some embodiments. For instance, stimulators 612 may be positioned at the STN or GPi regions of a patient’s brain. In some embodiments, stimulators 612 may be positioned at other areas of a patient’s brain, such as, but not limited to, ventral intermediate nucleus (VIM), nucleus accumbens (NAc), anterior nucleus of the thalamaus (ANT), periventricular/periaqueductal gray (PVG/PAG), centromedian nucleus (CMN) of the thalmus, posterior hypothalamus, cingulate cortex, medial forebrain bundle (MFB), zona incerta (ZI), pendunclopontine nucleus (PPN), and/or other regions of a patient’s brain. Stimulators 612 may be any type of stimulator, such as, but not limited to , electrodes, optical energy emitters, electromagnetic radiators, and/or other types of stimulators. In embodiments where stimulators 612 may be electrodes, monopolar, bipolar, directional, microelectrode, and/or other types of electrodes may be used. Contact points of electrodes may include about 4 to about 8 contact points, which may be arranged in a linear array, in some embodiments. Contact points may be made of biocompatible materials, such as, but not limited to, a platinum-iridium alloy. Insulation materials, such as silicone or other biocompatible polymers, may be used to insulate stimulators 612 and/or wire 608 from surrounding tissue.

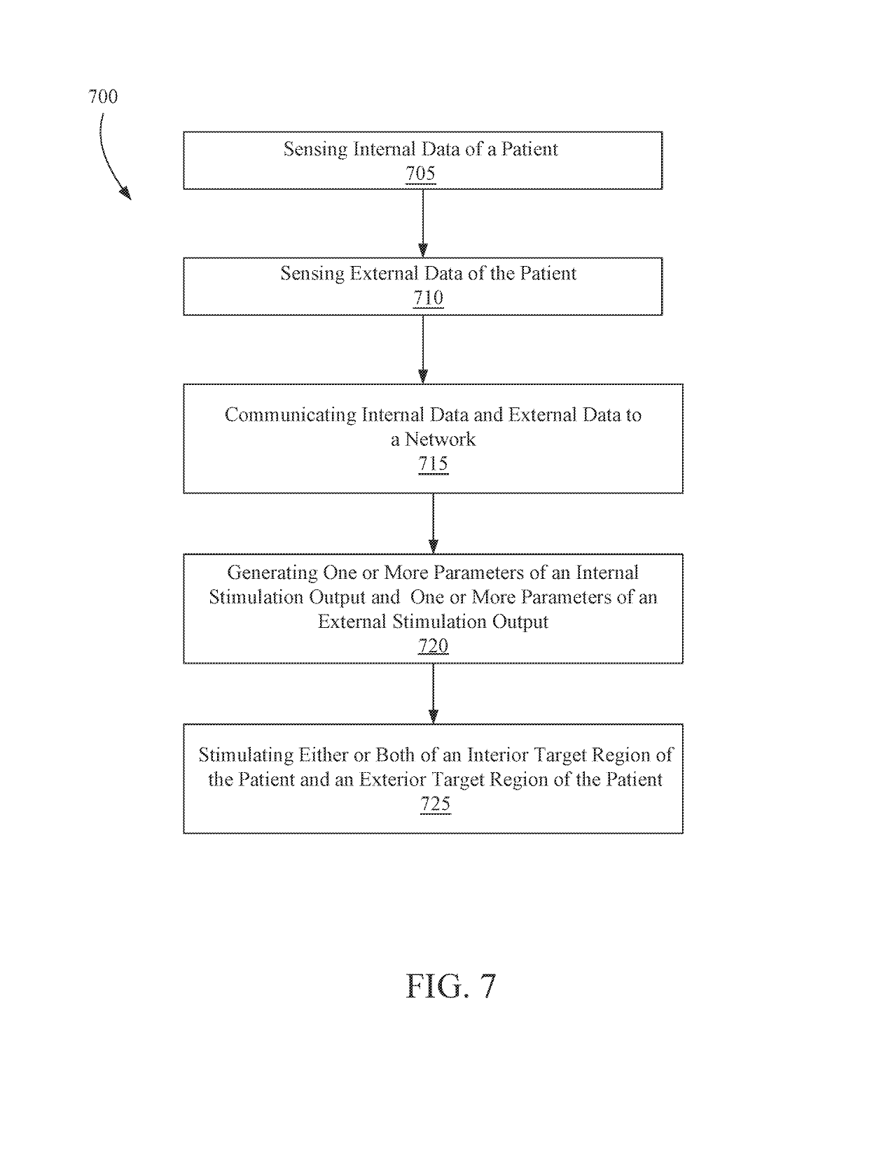

Referring now to FIG. 7, a method of neurostimulation 700 is presented. At step 705, method 700 includes sensing internal data of a patient. Internal data may be sensed by a sensor of an implantable neurostimulator. Internal data may include, but is not limited to, voltages, currents, local field potentials, and/or other data. This step may be implemented without limitation, as described above with reference to FIGS. 1-6.

At step 710, method 700 includes sensing external data of the patient. External data may be sensed by a sensor of a wearable medical device. External data may include, but is not limited to, accelerometer values, gyroscope values, inertial measurement unit values, and/or other data. This step may be implemented without limitation, as described above with reference to FIGS. 1-6.

At step 715, method 700 includes communicating internal data and external data to a network. Internal data may be communicated to a network by a wireless communication device of an implantable neurostimulator and external data may be communicated to the network by a wireless communication device of a wearable neurostimulator. A network may be a direct communication between an implantable neurostimulator and a wearable neurostimulator. In some embodiments, a network may include a connection with a central computing device. This step may be implemented without limitation, as described above with reference to FIGS. 1-6.

At step 720, method 700 includes generating one or more parameters of an internal stimulation output and one or more parameters of an external stimulation output. An internal stimulation output may include a waveform output provided by an internal stimulator of an implantable neurostimulator. An external stimulation output may include a waveform output of an external stimulator of a wearable neurostimulator. Parameters may include, but are not limited to, frequencies, pulse widths, amplitudes, duty cycles, peak-to-peak values, and/or any other parameter described herein. Parameters may be generated based on internal data, external data, and/or other data. For instance, the internal stimulation output may be generated based at least in part on external data. The external stimulation output may be generated based at least in part on the internal data. This step may be implemented without limitation, as described above with reference to FIGS. 1-6.

At step 725, method 700 includes stimulating either or both of an internal target area of the patient and an external target area of the patient. An internal target area may be a brain of a patient, in some embodiments. An external target area may be one or more nerves and/or tissues of a patient, such as proprioceptive nerves, dermatomes, and/or other target areas described herein. Stimulating an internal target area of the patient may include providing the internal stimulation output to the internal target area of the patient through an internal stimulator of an implantable neurostimulator. Stimulating an external target area of the patient may include providing the external stimulation output to the external target area of the patient through an external stimulator of a wearable neurostimulator. This step may be implemented without limitation, as described above with reference to FIGS. 1-6.

Referring to FIG. 8, an exemplary machine learning module 800 may perform machine learning process(es) and may be configured to perform various determinations, calculations, processes and the like as described herein using one or more machine learning processes. Machine learning module 800 may utilize training data 804. For instance, and without limitation, training data 804 may include a plurality of data entries, each entry representing a set of data elements that were recorded, received, and/or generated together. Training data 804 may include data elements that may be correlated by shared existence in a given data entry, by proximity in a given data entry, or the like. Multiple data entries in training data 804 may demonstrate one or more trends in correlations between categories of data elements. For instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 804 according to various correlations. Correlations may indicate causative and/or predictive links between categories of data elements, which may be modeled as relationships such as mathematical relationships by machine learning processes as described in further detail below. Training data 804 may be formatted and/or organized by categories of data elements. Training data 804 may, for instance, be organized by associating data elements with one or more descriptors corresponding to categories of data elements. As a nonlimiting example, training data 804 may include data entered in standardized forms by one or more individuals, such that entry of a given data element in a given field in a form may be mapped to one or more descriptors of categories. Elements in training data 804 may be linked to descriptors of categories by tags, tokens, or other data elements. Training data 804 may be provided in fixed-length formats, formats linking positions of data to categories such as comma-separated value (CSV) formats and/or self-describing formats. Self-describing formats may include, without limitation, extensible markup language (XML), JavaScript Object Notation (JSON), or the like, which may enable processes or devices to detect categories of data.

With continued reference to refer to FIG. 8, training data 804 may include one or more elements that are not categorized. Uncategorized data of training data 804 may include data that may not be formatted or containing descriptors for some elements of data. In some embodiments, machine learning algorithms and/or other processes may sort training data 804 according to one or more categorizations. Machine learning algorithms may sort training data 804 using, for instance, natural language processing algorithms, tokenization, detection of correlated values in raw data and the like. In some embodiments, categories of training data 804 may be generated using correlation and/or other processing algorithms. As a nonlimiting example, in a body of text, phrases making up a number “n” of compound words, such as nouns modified by other nouns, may be identified according to a statistically significant prevalence of n-grams containing such words in a particular order. For instance, an n-gram may be categorized as an element of language such as a “word” to be tracked similarly to single words, which may generate a new category as a result of statistical analysis. In a data entry including some textual data, a person’s name may be identified by reference to a list, dictionary, or other compendium of terms, permitting ad-hoc categorization by machine learning algorithms, and/or automated association of data in the data entry with descriptors or into a given format. The ability to categorize data entries in an automated fashion may enable the same training data 804 to be made applicable for two or more distinct machine learning algorithms as described in further detail below. Training data 804 used by machine learning module 800 may correlate any input data as described in this disclosure to any output data as described in this disclosure, without limitation.

Further referring to FIG. 8, training data 804 may be filtered, sorted, and/or selected using one or more supervised and/or unsupervised machine learning processes and/or models as described in further detail below. In some embodiments, training data 804 may be classified using training data classifier 816. Training data classifier 816 may include a classifier. A “classifier” as used in this disclosure is a machine learning model that sorts inputs into one or more categories. Training data classifier 816 may utilize a mathematical model, an artificial neural network, or a program generated by a machine learning algorithm. A machine learning algorithm of training data classifier 816 may include a classification algorithm. A “classification algorithm” as used herein is one or more computer processes that generate a classifier from training data. A classification algorithm may sort inputs into categories and/or bins of data. A classification algorithm may output categories of data and/or labels associated with the data. A classifier may be configured to output a datum that labels or otherwise identifies a set of data that may be clustered together. Machine learning module 800 may generate a classifier, such as training data classifier 816 using a classification algorithm. Classification may be performed using, without limitation, linear classifiers such as without limitation logistic regression and/or naive Bayes classifiers, nearest neighbor classifiers such ask-nearest neighbors classifiers, support vector machines, least squares support vector machines, fisher’s linear discriminant, quadratic classifiers, decision trees, boosted trees, random forest classifiers, learning vector quantization, and/or neural network-based classifiers. As a non-limiting example, training data classifier 816 may classify elements of training data to one or more parameters of a therapy regime.

Still referring to FIG. 8, machine learning module 800 may be configured to perform a lazy-learning process 820 which may include a “lazy loading” or “call-when-needed” process and/or protocol. A “lazy-learning process” may include a process in which machine learning is performed upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 804. Heuristic may include selecting some number of highest-ranking associations and/or training data 804 elements. Lazy learning may implement any suitable lazy learning algorithm, including without limitation a K-nearest neighbors algorithm, a lazy naive Bayes algorithm, or the like. Persons skilled in the art, upon reviewing the entirety of this disclosure, will be aware of various lazy-learning algorithms that may be applied to generate outputs as described herein, including lazy learning applications of machine learning algorithms as described in further detail below.

Still referring to FIG. 8, machine learning processes as described herein may be used to generate machine learning models 824. A “machine learning model” as used herein is a mathematical and/or algorithmic representation of a relationship between inputs and outputs, as generated using any machine learning process including without limitation any process as described above, and stored in memory. For instance, an input may be sent to machine learning model 824, which once created, may generate an output as a function of a relationship that was derived. For instance, and without limitation, a linear regression model, generated using a linear regression algorithm, may compute a linear combination of input data using coefficients derived during machine learning processes to calculate an output. As a further non-limiting example, machine learning model 824 may be generated by creating an artificial neural network, such as a convolutional neural network comprising an input layer of nodes, one or more intermediate layers, and an output layer of nodes. Connections between nodes may be created via the process of “training” the network, in which elements from a training data 804 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 8, machine learning algorithms may include supervised machine learning process 828. A “supervised machine learning process” as used herein is one or more algorithms that receive labelled input data and generate outputs according to the labelled input data. For instance, supervised machine learning process 828 may include sensor data as described above as inputs, parameters of stimulation output as outputs, and a scoring function representing a desired form of relationship to be detected between inputs and outputs. A scoring function may maximize a probability that a given input and/or combination of elements inputs is associated with a given output to minimize a probability that a given input is not associated with a given output. A scoring function may be expressed as a risk function representing an “expected loss” of an algorithm relating inputs to outputs, where loss is computed as an error function representing a degree to which a prediction generated by the relation is incorrect when compared to a given input-output pair provided in training data 804. Persons skilled in the art, upon reviewing the entirety of this disclosure, will be aware of various possible variations of at least a supervised machine learning process 828 that may be used to determine relation between inputs and outputs. Supervised machine learning processes may include classification algorithms as defined above.

Further referring to FIG. 8, machine learning processes may include unsupervised machine learning processes 832. An “unsupervised machine learning process” as used herein is a process that calculates relationships in one or more datasets without labelled training data. Unsupervised machine learning process 832 may be free to discover any structure, relationship, and/or correlation provided in training data 804. Unsupervised machine learning process 832 may not require a response variable. Unsupervised machine learning process 832 may calculate patterns, inferences, correlations, and the like between two or more variables of training data 804. In some embodiments, unsupervised machine learning process 832 may determine a degree of correlation between two or more elements of training data 804.

Still referring to FIG. 8, machine learning module 800 may be designed and configured to create a machine learning model 824 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of I divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model. Linear regression models may be generalized in an embodiment to polynomial regression models, whereby a polynomial equation (e.g. a quadratic, cubic or higher-order equation) providing a best predicted output/actual output fit is sought. Similar methods to those described above may be applied to minimize error functions, according to some embodiments.

Continuing to refer to FIG. 8, machine learning algorithms may include, without limitation, linear discriminant analysis. Machine learning algorithm may include quadratic discriminate analysis. Machine learning algorithms may include kernel ridge regression. Machine learning algorithms may include support vector machines, including without limitation support vector classification-based regression processes. Machine learning algorithms may include stochastic gradient descent algorithms, including classification and regression algorithms based on stochastic gradient descent. Machine learning algorithms may include nearest neighbors algorithms. Machine learning algorithms may include various forms of latent space regularization such as variational regularization. Machine learning algorithms may include Gaussian processes, such as Gaussian Process Regression. Machine learning algorithms may include cross-decomposition algorithms, including partial least squares and/or canonical correlation analysis. Machine learning algorithms may include naive Bayes methods. Machine learning algorithms may include algorithms based on decision trees, such as decision tree classification or regression algorithms. Machine learning algorithms may include ensemble methods such as bagging meta-estimator, forest of randomized tress, AdaBoost, gradient tree boosting, and/or voting classifier methods. Machine learning algorithms may include neural net algorithms, including convolutional neural net processes

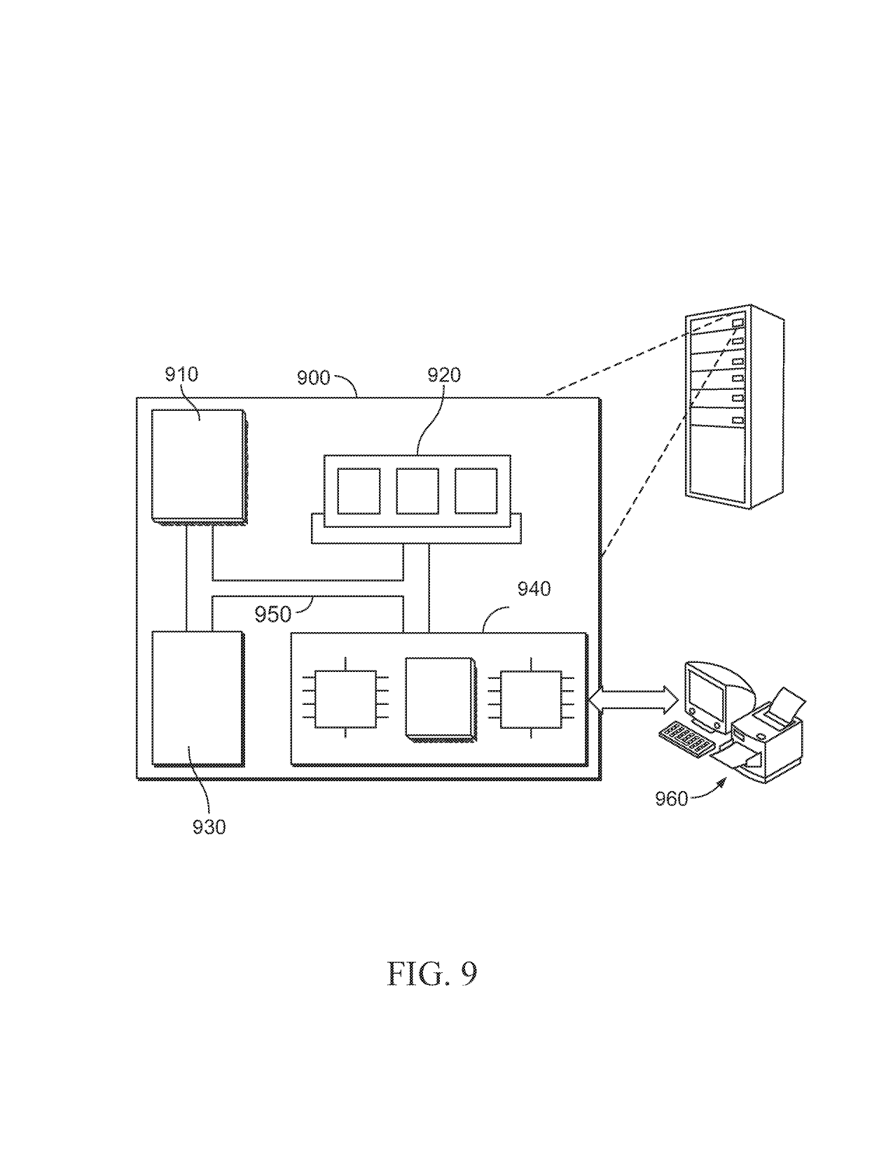

FIG. 9 is a block diagram of an example computer system 900 that may be used in implementing the technology described in this document. General-purpose computers, network appliances, mobile devices, or other electronic systems may also include at least portions of the system 900. The system 900 includes a processor 910, a memory 920, a storage device 930, and an input/output device 940. The apparatus may include disk storage and/or internal memory, each of which may be communicatively connected to each other. The apparatus 100 may include a processor 910. The processor 910 may enable both generic operating system (OS) functionality and/or application operations. In some embodiments, the processor 910 and the memory 920 may be communicatively connected. As used in this disclosure, “communicatively connected” means connected by way of a connection, attachment, or linkage between two or more elements which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components.

Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure. In some embodiments, the processor 910 may include any computing device as described in this disclosure, including without limitation a microcontroller, microprocessor, digital signal processor (DSP) and/or system on a chip (SoC) as described in this disclosure. The processor 910 may include, be included in, and/or communicate with a mobile device such as a mobile telephone or smartphone. The processor 910 may include a single computing device operating independently, or may include two or more computing device operating in concert, in parallel, sequentially or the like. Two or more computing devices may be included together in a single computing device or in two or more computing devices. The processor 910 may interface or communicate with one or more additional devices as described below in further detail via a network interface device.

Network interface device may be utilized for connecting the processor 910 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. The processor 910 may include but is not limited to, for example, a computing device or cluster of computing devices in a first location and a second computing device or cluster of computing devices in a second location. The processor 910 may include one or more computing devices dedicated to data storage, security, distribution of traffic for load balancing, and the like. The processor 910 may distribute one or more computing tasks as described below across a plurality of computing devices of computing device, which may operate in parallel, in series, redundantly, or in any other manner used for distribution of tasks or memory between computing devices. The processor 910 may be implemented using a “shared nothing” architecture in which data is cached at the worker, in an embodiment, this may enable scalability of system 900 and/or processor 910.

With continued reference to FIG. 9, processor 910 and/or a computing device may be designed and/or configured by memory 920 to perform any method, method step, or sequence of method steps in any embodiment described in this disclosure, in any order and with any degree of repetition. For instance, the processor 910 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. The processor 910 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Each of the components 910, 920, 930, and 940 may be interconnected, for example, using a system bus 950. The processor 910 is capable of processing instructions for execution within the system 900. In some implementations, the processor 910 is a single-threaded processor. In some implementations, the processor 910 is a multi-threaded processor. In some implementations, the processor 910 is a programmable (or reprogrammable) general purpose microprocessor or microcontroller. The processor 910 is capable of processing instructions stored in the memory 920 or on the storage device 930.

The memory 920 stores information within the system 900. In some implementations, the memory 920 is a non-transitory computer-readable medium. In some implementations, the memory 920 is a volatile memory unit. In some implementations, the memory 920 is a non-volatile memory unit.

The storage device 930 is capable of providing mass storage for the system 900. In some implementations, the storage device 930 is a non-transitory computer-readable medium. In various different implementations, the storage device 930 may include, for example, a hard disk device, an optical disk device, a solid-date drive, a flash drive, or some other large capacity storage device. For example, the storage device may store long-term data (e.g., database data, file system data, etc.). The input/output device 940 provides input/output operations for the system 900. In some implementations, the input/output device 940 may include one or more network interface devices, e.g., an Ethernet card, a serial communication device, e.g., an RS-232 port, and/or a wireless interface device, e.g., an 802.11 card, a 3G wireless modem, or a 4G/5G wireless modem. In some implementations, the input/output device may include driver devices configured to receive input data and send output data to other input/output devices, e.g., keyboard, printer and display devices 960. In some examples, mobile computing devices, mobile communication devices, and other devices may be used.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A neurostimulation system, comprising:
an implantable neurostimulator, the implantable neurostimulator comprising:
a first sensor configured to sense internal data relating to a symptom of a neurological movement disorder of a patient;
an internal stimulator configured to provide an internal stimulation output to an internal target area of the patient;
a first wireless communication device configured to communicate the internal data with a network; and
a first processor in communication with the first sensor, internal stimulator, and first wireless communication device, wherein the first processor is configured to receive the internal data from the first sensor and adjust the internal stimulation output based on the internal data; and
a wearable neurostimulator, the wearable neurostimulator comprising:
a second sensor configured to sense external data of the patient;
an external stimulator configured to provide an external stimulation output to an external target area of the patient;
a second wireless communication device configured to communicate the external data with the network and receive the internal data from the network; and
a second processor in communication with the second sensor, external stimulator, and second wireless communication device, wherein the second processor is configured to receive the external data from the second sensor and adjust the external stimulation output based at least in part on the internal data received from the network.

2. The neurostimulation system of claim 1, wherein the internal stimulation output and the external simulation output are adjusted simultaneously.

3. The neurostimulation system of claim 1, wherein the network is a direct architecture network between the first wireless communication device and the second wireless communication device.

4. The neurostimulation system of claim 3, wherein the second wireless communication device utilizes a beam forming technique to connect with the first wireless communication device.

5. The neurostimulation system of claim 1, wherein the first wireless communication comprises an antenna placed close to a surface of a housing of the implantable neurostimulator, the antenna configured to propagate electromagnetic radiation through tissue of the patient.

6. The neurostimulation system of claim 1, wherein the first wireless communication device receives external data from the wearable neurostimulator via the network.

7. The neurostimulation system of claim 6, wherein the first processor is further configured to adjust the internal stimulation output based on the internal data.

8. The neurostimulation system of claim 1, wherein the network is a central architecture, the central architecture comprising a central computing device in communication with both the first and second wireless communication devices, wherein the central computing device is configured to:
receive both the internal and external data; and
calculate one or more parameters of either or both of the internal stimulation output and the external stimulation output based on the internal and external data.

9. The neurostimulation system of claim 8, wherein the central computing device is further configured to calculate the one or more parameters of either or both of the internal stimulation output and the external stimulation output through a machine learning model.

10. The neurostimulation system of claim 1, wherein the implantable neurostimulator and the wearable neurostimulator form a closed-loop system.

11. A method of neurostimulation, comprising:
sensing, by a sensor of an implantable neurostimulator, internal data of the patient;
sensing, by a sensor of a wearable neurostimulator, external data of the patient;
communicating the internal data to the wearable neurostimulator and the external data to the implantable neurostimulator via a network in communication with both the wearable neurostimulator and the implantable neurostimulator;
generating, by a device in the network, one or more parameters of an internal stimulation output based on at least the external data and one or more parameters of an external stimulation output based on at least the internal data; and
stimulating either or both of an internal target area of the patient with the internal stimulation output through an internal stimulator of the implantable neurostimulator and an external target area of the patient with the external stimulation output through an external stimulator of the wearable neurostimulator.

12. The method of claim 11, wherein the device in the network is the implantable neurostimulator, wearable neurostimulator, or a central computing device.

13. The method of claim 11, wherein the network is a direct architecture network between the first wireless communication device and the second wireless communication device.

14. The method of claim 13, further comprising connecting the second wireless communication device to the first wireless communication device utilizing a beam forming technique.

15. The method of claim 11, wherein the first wireless communication comprises an antenna placed close to a surface of a housing of the implantable neurostimulator, the antenna configured to propagate electromagnetic radiation through tissue of the patient.

16. The method of claim 11, further comprising receiving, at the implantable neurostimulator, receiving external data from the wearable neurostimulator via the network.

17. The method of claim 16, further comprising adjusting, by the device in the network, the internal stimulation output based on the internal data.

18. The neurostimulation system of claim 1, wherein the network is a central architecture, the central architecture comprising a central computing device in communication with both the first and second wireless communication devices, wherein the method further comprises:
receiving, by the central computing device, both the internal and external data; and
calculating, by the central computing device, one or more parameters of either or both of the internal stimulation output and the external stimulation output based on the internal and external data.

19. The method of claim 18, further comprising calculating, by the computing device, the one or more parameters of either or both of the internal stimulation output and the external stimulation output through a machine learning model.

20. The method of claim 11, wherein the implantable neurostimulator and the wearable neurostimulator form a closed-loop system.
